SEQUENCE LISTING (56 sequences)

SEQ ID NO: 1 - DNA, 56 bp, E. Coli
acaacacattttactgctgtgaactttgttgtaatcctgcctgtgctggatgttat

SEQ ID NO: 2 - protein, 19 aa, E. Coli
NNTFYCCELCCNPACAGCY

SEQ ID NO: 3 - protein, 18 aa, Artificial Sequence
  ST active peptide #1
NTFYCCELCCYPACAGCN

SEQ ID NO: 4 - DNA, 56 bp, E. Coli
atagtagcaattactgctgtgaattgtgttgtaatcctgcttgtaacgggtgctat

SEQ ID NO: 5 - protein, 19 aa, E. Coli
NSSNYCCELCCNPACNGCY

SEQ ID NO: 6 - protein, 15 aa, Rattus Norvegious
PNTCEICAYAACTGC

SEQ ID NO: 7 - protein, 18 aa, Artificial Sequence
  ST active peptide #2
NNTFYCCELCCNPACAGC

SEQ ID NO: 8 - protein, 17 aa, Artificial Sequence
  ST active peptide #3
NTFYCCELCCNPACAGC

SEQ ID NO: 9 - protein, 16 aa, Artificial Sequence
  ST active peptide #4
TFYCCELCCNPACAGC

SEQ ID NO: 10 - protein, 15 aa, Artificial Sequence
  ST active peptide #5
FYCCELCCNPACAGC

SEQ ID NO: 11 - protein, 14 aa, Artificial Sequence
  ST active peptide #6
YCCELCCNPACAGC

SEQ ID NO: 12 - protein, 13 aa, Artificial Sequence
  ST active peptide #7
CCELCCNPACAGC

SEQ ID NO: 13 - protein, 18 aa, Artificial Sequence
  ST active peptide #8
NTFYCCELCCNPACAGCY

SEQ ID NO: 14 - protein, 17 aa, Artificial Sequence
  ST active peptide #9
TFYCCELCCNPACAGCY

SEQ ID NO: 15 - protein, 16 aa, Artificial Sequence
  ST active peptide #10
FYCCELCCNPACAGCY

SEQ ID NO: 16 - protein, 15 aa, Artificial Sequence
  ST active peptide #11
YCCELCCNPACAGCY

SEQ ID NO: 17 - protein, 14 aa, Artificial Sequence
  ST active peptide #12
CCELCCNPACAGCY

SEQ ID NO: 18 - protein, 17 aa, Artificial Sequence
  ST active peptide #13
NTFYCCELCCYPACAGC

SEQ ID NO: 19 - protein, 16 aa, Artificial Sequence
  ST active peptide #14
TFYCCELCCYPACAGC

SEQ ID NO: 20 - protein, 15 aa, Artificial Sequence
  ST active peptide #15
FYCCELCCYPACAGC

SEQ ID NO: 21 - protein, 14 aa, Artificial Sequence
  ST active peptide #16
YCCELCCYPACAGC

SEQ ID NO: 22 - protein, 13 aa, Artificial Sequence
  ST active peptide #17
CCELCCYPACAGC

SEQ ID NO: 23 - protein, 17 aa, Artificial Sequence
  ST active peptide #18
TFYCCELCCYPACAGCN

SEQ ID NO: 24 - protein, 16 aa, Artificial Sequence
  ST active peptide #19
FYCCELCCYPACAGCN

SEQ ID NO: 25 - protein, 15 aa, Artificial Sequence
  ST active peptide #20
YCCELCCYPACAGCN

SEQ ID NO: 26 - protein, 14 aa, Artificial Sequence
  ST active peptide #21
CCELCCYPACAGCN

SEQ ID NO: 27 - protein, 18 aa, artificial sequence
  ST Ib active peptide #1
NSSNYCCELCCNPACTGC

SEQ ID NO: 28 - protein, 17 aa, Artificial Sequence
  ST Ib active peptide #2
SSNYCCELCCNPACTGC

SEQ ID NO: 29 - protein, 16 aa, Artificial Sequence
  ST Ib active peptide #3
SNYCCELCCNPACTGC

SEQ ID NO: 30 - protein, 15 aa, Artificial Sequence
  ST Ib active peptide #4
NYCCELCCNPACTGC

SEQ ID NO: 31 - protein, 14 aa, Artificial Sequence
  ST Ib active peptide #5
YCCELCCNPACTGC

SEQ ID NO: 32 - protein, 13 aa, Artificial Sequence
  ST Ib active peptide #6
CCELCCNPACTGC

SEQ ID NO: 33 - protein, 18 aa, Artificial Sequence
  ST Ib active peptide #7
SSNYCCELCCNPACTGCY

SEQ ID NO: 34 - protein, 17 aa, Artificial Sequence
  ST Ib active peptide #8
SNYCCELCCNPACTGCY

SEQ ID NO: 35 - protein, 16 aa, Artificial Sequence
  ST Ib active peptide #9
NYCCELCCNPACTGCY

SEQ ID NO: 36 - protein, 15 aa, Artificial Sequence
  ST Ib active peptide #10
YCCELCCNPACTGCY

SEQ ID NO: 37 - protein, 14 aa, Artificial Sequence
  ST Ib active peptide #7
CCELCCNPACTGCY

SEQ ID NO: 38 - protein, 18 aa, Artificial Sequence
  ST active peptide #22
NTFYCCELCCNPACAGCY

SEQ ID NO: 39 - protein, 18 aa, Artificial Sequence
  ST active peptide #23
NTFYCCELCCAPACAGCY

SEQ ID NO: 40 - protein, 18 aa, Artificial Sequence
  ST active peptide #24
NTFYCCELCCNAACAGCY

SEQ ID NO: 41 - protein, 17 aa, Artificial Sequence
  ST active peptide #25
NTFYCCELCCNPACAGC

SEQ ID NO: 42 - protein, 15 aa, Artificial Sequence
  ST active peptide #26
YCCELCCNPACAGCY

SEQ ID NO: 43 - protein, 14 aa, Artificial Sequence
  ST active peptide #27
YCCELCCNPACAGC

SEQ ID NO: 44 - protein, 14 aa, Artificial Sequence
  ST active peptide #28
CCELCCNPACAGCY

SEQ ID NO: 45 - protein, 13 aa, Artificial Sequence
  ST Ib active peptide #29
CCELCCNPACAGC

SEQ ID NO: 46 - protein, 25 aa, Artificial Sequence
  ST receptor binding peptide #1
QACDPPSPPAEVCCDVCCNPACAGC

SEQ ID NO: 47 - protein, 16 aa, Artificial Sequence
  ST receptor binding peptide #2
IDCCICCNPACFGCLN

SEQ ID NO: 48 - protein, 18 aa, Artificial Sequence
  ST receptor binding peptide #3
SSDWDCCDVCCNPACAGC

SEQ ID NO: 49 - protein, 19 aa, Artificial Sequence
  E. Coli derivative 1
NSSNYCCELCCYPACTGCY

SEQ ID NO: 50 - protein, 13 aa, Artificial Sequence
  ST receptor binding peptide #2
CCDVCCNPACTGC

SEQ ID NO: 51 - protein, 14 aa, Artificial Sequence
  ST receptor binding peptide #3
CCDVCCYPACTGCY

SEQ ID NO: 52 - protein, 14 aa, Artificial Sequence
  ST receptor binding peptide #4
CCDLCCNPACAGCY

SEQ ID NO: 53 - protein, 14 aa, Artificial Sequence
  ST receptor binding peptide #5
CCQLCCNPACTGCY

SEQ ID NO: 54 - protein, 15 aa, Homo sapiens
PGTCEICAYAACTGC

SEQ ID NO: 55 - protein, 15 aa, Didelphis Virginiana
QEDCELCINVACTGC

SEQ ID NO: 56 - protein, 16 aa, artificial sequence
  Homo sapiens
NDDCELCVNVACTGCL

(19) United States
(12) Patent Application Publication
(10) Pub. No.: US 2014/0213534 A1
(43) Pub. Date: Jul. 31, 2014

(54) INTESTINAL HYPERPERMEABILITY AND PREVENTION OF SYSTEMIC DISEASE

(71) Applicants: Scott A. Waldman, Ardmore, PA (US); Adam E. Snook, Aston, PA (US); Peng Li, Salt Lake City, UT (US); Jieru Egeria Lin, Philadelphia, PA (US)

(72) Inventors: Scott A. Waldman, Ardmore, PA (US); Adam E. Snook, Aston, PA (US); Peng Li, Salt Lake City, UT (US); Jieru Egeria Lin, Philadelphia, PA (US)

(73) Assignee: Thomas Jefferson University, Philadelphia, PA (US)

(21) Appl. No.: 14/122,170

(22) PCT Filed: Apr. 27, 2012
(86) PCT No.: PCT/US2012/035492

§ 371 Date: Feb. 28, 2014

Related U.S. Application Data
(60) Provisional application No. 61/489,203, filed on May 23, 2011.

(51) Int. Cl. A61K 38/10; A61K 45/06

(52) U.S. Cl. CPC A61K 38/10; A61K 45/06
USPC 514/21.4; 514/21.5

(57) ABSTRACT

Compositions for and methods of preventing or reducing the severity intestinal hyperpermeabilization in an individual are disclosed. Compositions for and methods of preventing or reducing the severity of a disease or condition caused or exacerbated by intestinal hyperpermeabilization in an individual identified as being at risk of a disease or condition caused or exacerbated by intestinal hyperpermeabilization are also disclosed. Compositions for and methods of treating an individual who has been identified as having a disease or condition caused or exacerbated by intestinal hyperpermeabilization are additionally disclosed.

### FIELD OF THE INVENTION

The present invention relates to compositions for and methods of preventing intestinal hyperpermeability and associated systemic disease, including neoplasms, through guanylyl cyclase C-mediated intestinal barrier enhancement. According to the invention, cyclic guanosine monophosphate levels are modulated by activation of guanylyl cyclase C or other modalities to maintain intestinal integrity and to prevent and minimize disease and conditions which result due to hyperpermeability.

### BACKGROUND OF THE INVENTION

Typically the single layer of epithelium covering intestinal surfaces are fused together by tight junctions, forming a barrier to the free diffusion of most microorganisms, macromolecules, antigens, and metabolic and microbial toxins of the gut between epithelial cells. This prevents host exposure to these harmful molecules. However, a compromise in barrier fitness results intestinal hyperpermeability and allows some bacteria and their toxins, food products, environmental macromolecules, etc to access the blood stream. This directly compromises liver, lymphatic and immune homeostasis, as well disrupting other organ physiology, producing, or exacerbating, systemic pathology. Indeed, intestinal hyperpermeability (also known as Leaky Gut Syndrome) has been suggested to contribute to at least 50 percent of chronic health care complaints.

Possible links between breeches in the integrity of the intestinal barrier and numerous diseases and conditions have been noted or suggested. Viljoen M, et al. East Afr Med J. 80(6): 324-330, 2003 for example refer to gastrointestinal diseases, including, but not limited to, irritable bowel syndrome, Crohn's disease, ulcerative colitis, and celiac disease. Hamilton I, et al. Q J Med. 56: 559-67, 1985 have reported on dermatologic disorders, including, but not limited to, eczema, urticaria-angiedema, psoriasis and dermatitis herpetiformis while Miele L, et al. Hepatology. 49(6): 1877-87, 2009 and Viljoen M, et al. East Afr Med J. 80(6): 324-330, 2003 have studied hepatobiliary disorders, including, but not limited to, alcoholic and nonalcoholic liver disease, obstructive jaundice, extrahepatic cholestasis, chronic hepatitis. Krack A, et al. European Heart Journal. 26, 2368-2374, 2005 refer to cardiovascular disorders, including, but not limited to, chronic or congestive heart failure (CHF), while Anderson J, et al. Am J Clin Nutr 70: 307-308, 1999 refers to coronary heart disease. Pulmonary disorders, including, but not limited to, lung injury induced by ischemia/reperfusion are reported by Nakagawa H, et al, Surgery, 145 (1), 48-56, 2009), while pulmonary hypertension or hyperoxic lung injury are discussed in Arkovitz M, et al, Journal of Pediatric Surgery, 31(8), 1009-1015, 1996). Wallaert B, et al. Am Res Respir Dis. 145: 1440-45, 1992) refer to active pulmonary sarcoidosis. Neuropsychiatric disorders, including, but not limited to, autism as disclosed in Kidd P M. Altern Med Rev. 7(4): 292-316, 2002, schizophrenia as disclosed in Wood N C, et al. Br J Psychiatry. 150: 853, 1987, and seizures, migraine, sensory neuropathy, myasthenia gravis, cerebral vasculitis, multiple sclerosis, and depression Neuroendocrinology Letters Vol. 29 (1) 2008 have been studied. Systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus have been referred to in Viljoen M, et al. East Afr Med J. 80(6): 324-330, 2003 and Majamaa H, et al. J Allery Clin Immunol. 97:985-990, 1996 while other potential diseases including, but not limited to, acne, allergies, fibromyalgia, chronic fatigue syndrome, halitosis, insomnia, nutritional deficiencies, and AIDS are mentioned in Miller, A L, et al. Alt Med Review 2(5):330-345, 1997.

In addition, intestinal hyperpermeability may be linked to local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer. Environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals are exacerbated and genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome, and autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis may all have possible links as well.

Han X, Mann E A, Gilbert S, Guan Y, Montrose M H, Cohen M B. M1696 Novel Role of Guanylyl Cyclase C (GCC) in Maintenance of Intestinal Barrier Function. Gastroenterology 2009; 136:A-412 disclose that mice lacking the intestinal cellular receptor guanylyl cyclase C (GCC) had increased intestinal permeability compared to GCC+ mice and that tight junctions at the crypts but not villi were disrupted in the GCC deficient mice.

There remains a need for treatments to prevent and treat intestinal hyperpermeability. There remains a need to prevent and treat systemic or peripheral disease by improving and maintaining intestinal barrier integrity.

### SUMMARY OF THE INVENTION

The present invention relates to methods of preventing or reducing the severity intestinal hyperpermeabilization in an individual comprising the step of administering to the individual an effective amount of one or more compounds that elevates intracellular cGMP levels in intestinal cells.

The present invention also relates to compositions for preventing or reducing the severity intestinal hyperpermeabilization in an individual.

The present invention further relates to methods of preventing or reducing the severity of a disease or condition caused or exacerbated by intestinal hyperpermeabilization in an individual identified as being at risk of a disease or condition caused or exacerbated by intestinal hyperpermeabilization. The methods comprise the step of administering to the individual an amount of one or more compounds that elevates intracellular cGMP levels in intestinal cells sufficient prevent or reduce intestinal hyperpermeabilization by an amount sufficient to prevent or reduce the severity of the disease or condition caused or exacerbated by intestinal hyperpermeabilization.

The present invention also relates to compositions for preventing or reducing the severity of a disease or condition caused or exacerbated by intestinal hyperpermeabilization.

The present invention further relates to methods of treating an individual who has been identified as having a disease or condition caused or exacerbated by intestinal hyperpermeabilization. The methods comprise the step of administering to the individual an amount of one or more compounds that elevates intracellular cGMP levels in intestinal cells sufficient prevent or reduce intestinal hyperpermeabilization by an amount sufficient to prevent or reduce the severity of the disease or condition caused or exacerbated by intestinal hyperpermeabilization.

The present invention also relates to compositions for treating an individual who has a disease or condition caused or exacerbated by intestinal hyperpermeabilization.

### DESCRIPTION OF PREFERRED EMBODIMENTS

### Definitions

As used herein the terms “guanylyl cyclase C agonist” and “GCC agonists” are used interchangeably and refer to molecules which bind to guanylyl cyclase C on a cell surface and thereby induce its activity which results in cGMP accumulation within the cell.

As used herein the terms “soluble guanylyl cyclase activator” and “sGC activator” are used interchangeably and refer to molecules which bind to soluble guanylyl cyclase and thereby induce its activity which results in cGMP accumulation within the cell.

As used herein the terms “phosphodiesterase inhibitor” and “PDE inhibitors” are used interchangeably and refer to molecules which inhibit the activity of one or more forms or subtypes of the cGMP-hydrolyzing phosphodiesterase enzyme and thereby bringing about cGMP accumulation within the cell.

As used herein the terms “multidrug resistance-associated protein inhibitors” and “MRP inhibitors” are used interchangeably and refer to molecules which inhibit the activity of one or more forms or subtypes of the cGMP-transporting MRPs and thereby bringing about cGMP accumulation within the cell.

As used herein the term “effective amount” refers to the amount of compound(s) effective to reduce or prevent hyperpermeability of the intestinal barrier. In some embodiments, a therapeutically effective amount of a compound used to treat or prevent systemic or peripheral disease is an amount of compound(s) to reduce or prevent hyperpermeability of the intestinal barrier sufficient to treat or prevent systemic or peripheral disease.

### Intestinal Barrier Integrity

The maintenance of intestinal barrier integrity has important implications beyond the prevention of local bacterial invasion and inflammation. Indeed, barrier disruption can lead to bacterial translocation, systemic genotoxicity, infection, and hypersensitivity, aging, tumorigenesis in extra-intestinal tissues and deterioration of pre-existent pathophysiological conditions. Identification of the intestine-specific receptor, GCC, as a key regulator of intestinal barrier integrity and guardian of not only intestinal, but also extra-intestinal, homeostasis and genomic integrity identifies GCC stimulation, using GCC agonist administration, or other approaches, as convenient (oral) and targeted (intestine-specific) approaches to preventing or reducing disease and conditions, particularly, in those individuals who have been identified as being at a higher than normal risk of developing such disease or condition. GCC ligand prophylaxis increases barrier function, preventing intestinal hyperpermeability, and may prevent systemic toxicity associated with intestinal contents, reducing the incidence of intestinal hyperpermeability-associated diseases, including hypersensitivity, systemic cancers, etc in humans and animals, decreasing the progression of aging and preventing mortality propagated by intestinal hyperpermeability. Currently, no similar approach exists.

In animal models, intestinal barrier disruption and inflammation allows environmental molecules and the normal microflora to penetrate host tissues and induce toxicity, indolent inflammation and genotoxic stress outside of the gastrointestinal tract. Importantly, mice deficient in the intestine-specific protein, guanylyl cyclase C (GCC), were found to have intestinal barrier disruption associated with significantly increased colitis, bacterial translocation from intestine into the systemic compartment, systemic genotoxicity and spontaneous tumor formation in extra-intestinal tissues.

Administration of GCC agonists decreased intestinal permeability in animal models that express GCC. Because GCC-signaling can be modulated by the gut hormones guanylin and uroguanylin, oral delivery of these hormones (or analogues), or other ways of stimulating GCC activity in intestine, may be used as an extra-intestinal immune modulator, cancer chemopreventative, and suppressor of multi-organ pathophysiology by increasing intestinal barrier integrity and decreasing systemic pathology associated with intestinal hyperpermeability.

cGMP

The intracellular accumulation of cGMP helps the cell maintain genomic integrity by enhanced DNA damage sensing and repair for a period sufficient to reduce cell damage.

The intracellular increase of cGMP levels enhances p53 mediated cell survival in the intestine. Thus, increasing intracellular cGMP levels in intestinal cells protects intestinal epithelial cells. cGMP levels must be increased to an amount effective to enhance p53 mediated cell survival.

The use of compounds which increase cGMP productions and/or compounds which inhibit cGMP degradation or export from the cell result in an increase in cGMP levels. When administered to the normal GI tract, the increase in cGMP levels serves to protect the cells from cell death which is associated with side effects associated with chemotherapy and radiation therapy, thereby increasing safety of these therapies. In addition, the reduction of side effects allows for toleration of increasing and more effective doses. When delivered to cancer cells such as lung, breast, prostate, colorectal, and liver cancers in order to increase cGMP levels, the cancer cells may become more susceptible to chemotherapy and radiation therapy thereby increasing the efficacy of the treatment.

Compounds which increase cGMP production include activators of guanylyl cyclase C (GCC).

Compounds which inhibit cGMP degradation and/or export from the include phosphodiesterase enzyme (PDE) inhibitors which inhibit PDE forms and subtypes involved in converting cGMP.

Compounds which inhibit cGMP export from the cell include multidrug resistance protein (MRP) inhibitors which inhibit MRP forms and subtypes involved in transport of cGMP.

These compounds can be used alone or in combinations of two or more to increase intracellular cGMP levels to protect cells of the intestines from cell death associated with chemotherapy and radiation therapy side effects and may render cancer cells more susceptible to cell death.

### GCC

GCC is the predominant guanylyl cyclase in the GI tract. Accordingly, the use of GCC activators or agonists is particularly effective to increase intracellular cGMP in the GI tract. The GCC activators include endogenous peptides guanylin and uroguanylin as well as heat stable enterotoxins produced by bacteria, such as E. coli STs. PDE inhibitors and MRP inhibitors are also known. In some embodiments, one or more GCC agonists is used. In some embodiments, one or more PDE inhibitors is used. In some embodiments, one or more MRP inhibitors is used. In some embodiments, a combination of one or more GCC agonists and/or one or more PDE inhibitors and/or one or more MRP inhibitors is used.

Activation of the cellular receptor guanylyl cyclase C (GCC), a protein expressed primarily in the GI tract, protects cells in the GI tract. The activation of GCC leads to intracellular accumulation of cGMP. By activating GCC, intracellular cGMP levels are increased and the health of the cells and integrity of the barrier is maintained.

GCC is the intestinal epithelial cell receptor for the endogenous paracrine hormones guanylin and uroguanylin. Diarrheagenic bacterial heat-stable enterotoxins (STs) also target GCC. Hormone-receptor interaction between guanylin or uroguanylin and the extracellular domain of GCC (also referred to as ST receptor) and the interaction between the peptide enterotoxin ST and the extracellular domain of GCC each activates the intracellular catalytic domain of GCC which converts GTP to cyclic GMP (cGMP). This cyclic nucleotide, as a second messenger, activates its downstream effectors mediating GCC's cellular effects. Increasing intracellular cGMP by activating guanylyl cyclase (including particulate and soluble forms) or by inhibiting cGMP degradation or expulsion by inhibitors of phosphodiesterases (PDEs) or multi-drug resistance associated proteins (MRPs), respectively, prevents the death of normal intestinal epithelial cells.

Increases in cGMP levels such as those increases associated with GCC activation protect intestinal cells. Thus, activation of GCC can be effected such that the GCC activated intestinal cells are better able to maintain the tight junctions of the intestinal barrier. In addition to activation of GCC, protection of intestinal epithelial cells can be undertaken by increasing cGMP levels. The level of GCC activation or other increase in cGMP levels must be sufficient to enhance cell survival.

Administration of a GCC agonist refers to administration of one or more compounds that bind to and activate GCC.

Guanylyl cyclase C (GCC) is a cellular receptor expressed by cells lining the large and small intestines. The binding of GCC agonists to GCC in the gastrointestinal track is known to activate GCC, leading to an increase in intracellular cGMP, which results in activation of downstream signaling events.

### GCC Agonists

GCC agonists are known. Two native GCC agonists, guanylin and uroguanylin, have been identified (see U.S. Pat. Nos. 5,969,097 and 5,489,670, which are each incorporated herein by reference. In addition, several small peptides, which are produced by enteric pathogens, are toxigenic agents which cause diarrhea (see U.S. Pat. No. 5,518,888, which is incorporated herein by reference). The most common pathogen derived GCC agonist is the heat stable entertoxin produced by strains of pathogenic E. coli. Native heat stable enterotoxin produced by pathogenic E. coli is also referred to as ST. A variety of other pathogenic organisms including Yersinia and Enterobacter, also make enterotoxins which can bind to guanylyl cyclase C in an agonistic manner. In nature, the toxins are generally encoded on a plasmid which can “jump” between different species. Several different toxins have been reported to occur in different species. These toxins all possess significant sequence homology, they all bind to ST receptors and they all activate guanylate cyclase, producing diarrhea.

ST has been both cloned and synthesized by chemical techniques. The cloned or synthetic molecules exhibit binding characteristics which are similar to native ST. Native ST isolated from E. coli is 18 or 19 amino acids in length. The smallest “fragment” of ST which retains activity is the 13 amino acid core peptide extending toward the carboxy terminal from cysteine 6 to cysteine 18 (of the 19 amino acid form). Analogues of ST have been generated by cloning and by chemical techniques. Small peptide fragments of the native ST structure which include the structural determinant that confers binding activity may be constructed. Once a structure is identified which binds to ST receptors, non-peptide analogues mimicking that structure in space are designed.

U.S. Pat. Nos. 5,140,102 and 7,041,786, and U.S. Published Applications US 2004/0258687 A1 and US 2005/0287067 A1 also refer to compounds which may bind to and activate guanylyl cyclase C.

SEQ ID NO:1 discloses a nucleotide sequence which encodes 19 amino acid ST, designated ST Ia, reported by So and McCarthy (1980) Proc. Natl. Acad. Sci. USA 77:4011, which is incorporated herein by reference.

The amino acid sequence of ST Ia is disclosed in SEQ ID NO:2.

SEQ ID NO:3 discloses the amino acid sequence of an 18 amino acid peptide which exhibits ST activity, designated ST 1*, reported by Chan and Giannella (1981) J. Biol. Chem. 256:7744, which is incorporated herein by reference.

SEQ ID NO:4 discloses a nucleotide sequence which encodes 19 amino acid ST, designated ST Ib, reported by Mosely et al. (1983) Infect. Immun. 39:1167, which is incorporated herein by reference.

The amino acid sequence of ST Ib is disclosed in SEQ ID NO:5.

A 15 amino acid peptide called guanylin which has about 50% sequence homology to ST has been identified in mammalian intestine (Currie, M. G. et al. (1992) Proc. Natl. Acad Sci. USA 89:947-951, which is incorporated herein by reference). Guanylin binds to ST receptors and activates guanylate cyclase at a level of about 10- to 100-fold less than native ST. Guanylin may not exist as a 15 amino acid peptide in the intestine but rather as part of a larger protein in that organ. The amino acid sequence of guanylin from rodent is disclosed as SEQ ID NO:6.

SEQ ID NO:7 is an 18 amino acid fragment of SEQ ID NO:2. SEQ ID NO:8 is a 17 amino acid fragment of SEQ ID NO:2. SEQ ID NO:9 is a 16 amino acid fragment of SEQ ID NO:2. SEQ ID NO:10 is a 15 amino acid fragment of SEQ ID NO:2. SEQ ID NO:11 is a 14 amino acid fragment of SEQ ID NO:2. SEQ ID NO:12 is a 13 amino acid fragment of SEQ ID NO:2. SEQ ID NO:13 is an 18 amino acid fragment of SEQ ID NO:2. SEQ ID NO:14 is a 17 amino acid fragment of SEQ ID NO:2. SEQ ID NO:15 is a 16 amino acid fragment of SEQ ID NO:2. SEQ ID NO:16 is a 15 amino acid fragment of SEQ ID NO:2. SEQ ID NO:17 is a 14 amino acid fragment of SEQ ID NO:2.

SEQ ID NO:18 is a 17 amino acid fragment of SEQ ID NO:3. SEQ ID NO:19 is a 16 amino acid fragment of SEQ ID NO:3. SEQ ID NO:20 is a 15 amino acid fragment of SEQ ID NO:3. SEQ ID NO:21 is a 14 amino acid fragment of SEQ ID NO:3. SEQ ID NO:22 is a 13 amino acid fragment of SEQ ID NO:3. SEQ ID NO:23 is a 17 amino acid fragment of SEQ ID NO:3. SEQ ID NO:24 is a 16 amino acid fragment of SEQ ID NO:3. SEQ ID NO:25 is a 15 amino acid fragment of SEQ ID NO:3. SEQ ID NO:26 is a 14 amino acid fragment of SEQ ID NO:3.

SEQ ID NO:27 is an 18 amino acid fragment of SEQ ID NO:5. SEQ ID NO:28 is a 17 amino acid fragment of SEQ ID NO:5. SEQ ID NO:29 is a 16 amino acid fragment of SEQ ID NO:5. SEQ ID NO:30 is a 15 amino acid fragment of SEQ ID NO:5. SEQ ID NO:31 is a 14 amino acid fragment of SEQ ID NO:5. SEQ ID NO:32 is a 13 amino acid fragment of SEQ ID NO:5. SEQ ID NO:33 is an 18 amino acid fragment of SEQ ID NO:5. SEQ ID NO:34 is a 17 amino acid fragment of SEQ ID NO:5. SEQ ID NO:35 is a 16 amino acid fragment of SEQ ID NO:5. SEQ ID NO:36 is a 15 amino acid fragment of SEQ ID NO:5. SEQ ID NO:37 is a 14 amino acid fragment of SEQ ID NO:5.

SEQ ID NO:27, SEQ ID NO:31, SEQ ID NO:36 AND SEQ ID NO:37 are disclosed in Yoshimura, S., et al. (1985) FEBS Lett. 181:138, which is incorporated herein by reference.

SEQ ID NO:38, SEQ ID NO:39 and SEQ ID NO:40, which are derivatives of SEQ ID NO:3, are disclosed in Waldman, S. A. and O'Hanley, P. (1989) Infect. Immun. 57:2420, which is incorporated herein by reference.

SEQ ID NO:41, SEQ ID NO:42, SEQ ID NO:43, SEQ ID NO:44 and SEQ ID NO:45, which are a derivatives of SEQ ID NO:3, are disclosed in Yoshimura, S., et al. (1985) FEBS Lett. 181:138, which is incorporated herein by reference.

SEQ ID NO:46 is a 25 amino acid peptide derived from Y. enterocolitica which binds to the ST receptor.

SEQ ID NO:47 is a 16 amino acid peptide derived from V. cholerae which binds to the ST receptor. SEQ ID NO:47 is reported in Shimonishi, Y., et al. FEBS Lett. 215:165, which is incorporated herein by reference.

SEQ ID NO:48 is an 18 amino acid peptide derived from Y. enterocolitica which binds to the ST receptor. SEQ ID NO:48 is reported in Okamoto, K., et al. Infec. Immun. 55:2121, which is incorporated herein by reference.

SEQ ID NO:49, is a derivative of SEQ ID NO:5.

SEQ ID NO:50, SEQ ID NO:51, SEQ ID NO:52 and SEQ ID NO:53 are derivatives. SEQ ID NO:54 is the amino acid sequence of guanylin from human.

A 15 amino acid peptide called uroguanylin has been identified in mammalian intestine from opossum (Hamra, S. K. et al. (1993) Proc. Natl. Acad Sci. USA 90:10464-10468, which is incorporated herein by reference; see also Forte L. and M. Curry 1995 FASEB 9:643-650; which is incorporated herein by reference). SEQ ID NO:55 is the amino acid sequence of uroguanylin from opossum.

A 16 amino acid peptide called uroguanylin has been identified in mammalian intestine from human (Kita, T. et al. (1994) Amer. J. Physiol. 266:F342-348, which is incorporated herein by reference; see also Forte L. and M. Curry 1995 FASEEB 9:643-650; which is incorporated herein by reference). SEQ ID NO:56 is the amino acid sequence of uroguanylin from human.

SEQ ID NO:57 is the amino acid sequence of proguanylin, a guanylin precursor which is processed into active guanylin.

SEQ ID NO:58 is the amino acid sequence of prouroguanylin, a uroguanylin precursor which is processed into active uroguanylin.

Although proguanylin and prouroguanylin are precursors for mature guanylin and mature uroguanylin respectively, they may be used as GCC agonists as described herein provide they are delivered such that they can be processed into the mature peptides.

U.S. Pat. Nos. 5,140,102, 7,041,786 and 7,304,036, and U.S. Published Applications US 2004/0258687, US 2005/0287067, 20070010450, 20040266989, 20060281682, 20060258593, 20060094658, 20080025966, 20030073628, 20040121961 and 20040152868, which are each incorporated herein by reference, also refer to compounds which may bind to and activate guanylyl cyclase C.

In addition to human guanylin and human uroguanylin, guanylin or uroguanylin may be isolated or otherwise derived from other species such as cow, pig, goat, sheep, horse, rabbit, bison, etc. Such guanylin or uroguanylin may be administered to individuals including humans.

Antibodies including GCC binding antibody fragments can also be GCC agonists. Antibodies may include for example polyclonal and monoclonal antibodies including chimeric, primatized, humanized or human monoclonal antibodies as well as antibody fragments that bind to GCC with agonist activity such as CDRs, FAbs, F(Ab), Fv's including single chain Fv and the like. Antibodies may be IgE, IgA or IgM for example.

To improve intestinal barrier integrity, GCC agonists may be delivered to the colorectal track by the oral delivery of such GCC agonists. ST peptides and the endogenous GCC agonist peptides, for example, are stable and can survive the stomach acid and pass through the small intestine to the colorectal track. Sufficient dosages are provided to ensure that GCC agonist reaches the large intestine in sufficient quantities to induce accumulation of cGMP in those cells as well.

GCC agonists such as for example ST, guanylin and uroguanylin, can survive the gastric environment. Thus, they may be administered without coating or protection against stomach acid. However, in order to more precisely control the release of GCC agonists administered orally, the GCC agonist may be enterically coated so that some or all of the GCC agonist is released after passing through the stomach. Such enteric coating may also be designed to provide a sustained or extended release of the GCC agonist over the period of time with which the coated GCC agonist passes through the intestines. In some embodiments, the GCC agonist may be formulated to ensure release of some compound upon entering the large intestine. In some embodiments, the GCC agonist may be delivered rectally.

Most enteric coatings are intended to protect contents from stomach acid. Accordingly, they are designed to release active agent upon passing through the stomach. The coatings and encapsulations used herein are provided to begin releasing the GCC agonist in the small intestine and preferably over an extended period of time so that GCC agonist concentrations can be maintained t an effective level for a greater period of time.

According to some embodiments, the GCC agonists are coated or encapsulated with a sufficient amount of coating material that the time required for the coating material to dissolve and release the GCC agonists corresponds with the time required for the coated or encapsulated composition to travel from the mouth to intestines.

According to some embodiments, the GCC agonists are coated or encapsulated with coating material that does not fully dissolve and release the GCC agonists until it comes in contact with conditions present in the small intestine. Such conditions may include the presence of enzymes in the colorectal track, pH, tonicity, or other conditions that vary relative to the stomach.

According to some embodiments, the GCC agonists are coated or encapsulated with coating material that is designed to dissolve in stages as it passes from stomach to small intestine to large intestine.

According to some embodiments, the GCC agonists are complexed with another molecular entity such that they are inactive until the GCC agonists cease to be complexed with molecular entity and are present in active form. In such embodiments, the GCC agonists are administered as “prodrugs” which become processed into active GCC agonists in the colorectal track.

Examples of technologies which may be used to formulate GCC agonists for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

Examples of technologies which may be used to formulate GCC agonists or inducers for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference.

In some embodiments, the effective amount is delivered so that sufficient accumulation of cGMP results. Multiple doses may be administered to maintain levels such that the amount of GCC agonist present, either free or bound to GCC, remains ay or above the effective dose. In some embodiments, an initial loading dose and/or multiple administrations are required for cells of the intestine to become protected.

In some embodiments, GCC agonists which are peptides may be administered in an amount ranging from 100 ug to 1 gram every 4-48 hours. In some embodiments, GCC agonists are administered in an amount ranging from 1 mg to 750 mg every 4-48 hours. In some embodiments, GCC agonists are administered in an amount ranging from 10 mg to 500 mg every 4-48 hours. In some embodiments, GCC agonists are administered in an amount ranging from 50 mg to 250 mg every 4-48 hours. In some embodiments, GCC agonists are administered in an amount ranging from 75 mg to 150 mg every 4-48 hours,

In some embodiments, doses are administered every 4 or more hours. In some embodiments, doses are administered every 6 or more hours. In some embodiments, doses are administered every 8 or more hours. In some embodiments, doses are administered every 12 or more hours. In some embodiments, doses are administered every 24 or more hours. In some embodiments, doses are administered every 48 or more hours. In some embodiments, doses are administered every 4 hours or less. In some embodiments, doses are administered every 6 hours or less. In some embodiments, doses are administered every 8 hours or less. In some embodiments, doses are administered every 12 hours or less. In some embodiments, doses are administered every 24 hours or less. In some embodiments, doses are administered every 48 hours or less.

In some embodiments, additives or co-agents are administered in combination with GCC agonists to a minimize diarrhea or cramping/intestinal contractions-increased motity. For example, the individual may be administered a compound that before, simultaneously or after administration with a compound that relieves diarrhea. Such anti-diarrheal component may be incorporated in the formulation. Anti-diarrheal compounds and preparations, such as loperamide, bismuth subsalicylate and probiotic treatments such as strains of Lactobaccilus, are well known and widely available.

According to some aspects of the invention, innocuous bacteria of species that normally populate the colon are provided with genetic information needed to produce a guanylyl cyclase C agonist in the colon, making such guanylyl cyclase C agonist available to produce the effect of activating the guanylyl cyclase C on colon cells. The existence of a population of bacteria which can produce guanylyl cyclase C agonist provides a continuous administration of the guanylyl cyclase C agonist. In some embodiments, the nucleic acid sequences that encode the guanylyl cyclase C agonist may be under the control of an inducible promoter. Accordingly, the individual may turn expression on or off depending upon whether or not the inducer is ingested. In some embodiments, the inducer is formulated to be specifically released in the colon, thereby preventing induction of expression by the bacteria that may be populating other sites such as the small intestine. In some embodiments, the bacteria are is sensitive to a particular drug or auxotrophic such that it can be eliminated by administration of the drug or withholding an essential supplement.

The technology for introducing expressible forms of genes into bacteria is well known and the materials needed are widely available.

In some embodiments, bacteria which comprise coding sequences for a GCC agonist may be those of a species which commonly inhabits the intestinal track of an individual. Common gut flora include species from the genera Bacteroides, Clostridium, Fusobacterium, Eubacterium, Ruminococcus, Peptococcus, Peptostreptococcus, Bifidobacteriu, Escherichia and Lactobacillus. In some embodiments, the bacteria is selected from a strain known to be useful as a probiotic. Examples of species of bacteria used as compositions for administration to humans include Bifidobacterium bifidum; Escherichia coli, Lactobacillus acidophilus, Lactobacillus rhamnosus, Lactobacillus casei, and Lactobacillus johnsonii. Other species include Lactobacillus bulgaricus, Streptococcus thermophilus, Bacillus coagulans and Lactobacillus bifidus. Examples of strains of bacteria used as compositions for administration to humans include: B. infantis 35624, (Align); Lactobacillus plantarum 299V; Bifidobacterium animalis DN-173 010; Bifidobacterium animalis DN 173 010 (Activia Danone); Bifidobacterium animalis subsp. lactis BB-12 (Chr.Hansen); Bifidobacterium breve Yakult Bifiene Yakult; Bifidobacterium infantis 35624 Bifidobacterium lactis HNO19 (DR10) Howaru™ Bifido Danisco; Bifidobacterium longum BB536; Escherichia coli Nissle 1917; Lactobacillus acidophilus LA-5 Chr. Hanse; n; Lactobacillus acidophilus NCFM Rhodia Inc.; Lactobacillus casei DN114-001; Lactobacillus casei CRL431 Chr. Hansen; Lactobacillus casei F19 Cultura Arla Foods; Lactobacillus casei Shirota Yakult Yakult; Lactobacillus casei immunitass Actimel Danone; Lactobacillus johnsonnii La1 (=Lactobacillus LC1) Nestlé; Lactobacillus plantarum 299V ProViva Probi IBS; Lactobacillus reuteri ATTC 55730 BioGaia Biologics; Lactobacillus reuteri SD2112; Lactobacillus rhamnosus ATCC 53013 Vifit and others Valio; Lactobacillus rhamnosus LB21 Verum Norrmejerier; Lactobacillus salivarius UCC118; Lactococcus lactis L1A Verum Norrmejerier; Saccharomyces cerevisiae (boulardii) lyo; Streptococcus salivarius ssp thermophilus; Lactobacillus rhamnosus GR-1; Lactobacillus reuteri RC-14; Lactobacillus acidophilus CUL60; Bifidobacterium bifidum CUL 20; Lactobacillus helveticus R0052; and Lactobacillus rhamnosus R0011.

The following U.S. patents, which are each incorporated herein by reference, disclose non-pathogenic bacteria which can be administered to individuals. U.S. Pat. No. 6,200,609; U.S. Pat. No. 6,524,574, U.S. Pat. No. 6,841,149, U.S. Pat. No. 6,878,373, U.S. Pat. No. 7,018,629, U.S. Pat. No. 7,101,565, U.S. Pat. No. 7,122,370, U.S. Pat. No. 7,172,777, U.S. Pat. No. 7,186,545, U.S. Pat. No. 7,192,581, U.S. Pat. No. 7,195,906, U.S. Pat. No. 7,229,818, and U.S. Pat. No. 7,244,424.

Accordingly the aspects of the invention, bacteria would first be provided with genetic material encoding a GCC agonist in a form that would permit expression 1e of the agonist peptide within the bacteria, either constitutively or upon induction by the presence of an inducer that would turn on an inducible promoter.

Some embodiments comprise inducible regulatory elements such as inducible promoters. Typically, an inducible promoter is one in which an agent, when present, interacts with the promoter such that expression of the coding sequence operably linked to the promoter proceeds. Alternatively, an inducible promoter can include a repressor which is an agent that interacts with the promoter and prevent expression of the coding sequence operably linked to the promoter. Removal of the repressor results in expression of the coding sequence operably linked to the promoter.

The agents that induce an inducible promoter are preferably not naturally present in the organism where expression of the transgene is sought. Accordingly, the transgene is only expressed when the organism is affirmatively exposed to the inducing agent. Thus, in a bacterium that includes a transgene operably linked to an inducible promoter, when the bacterium is living within the gut of an individual, the promoter may be turned on and the transgene expressed when the individual ingests the inducing agent.

The agents that induce an inducible promoter are preferably not toxic. Thus, in a bacterium that includes a transgene operably linked to an inducible promoter, the inducing agent is preferably not toxic to the individual in whose gut the bacterium is living such that when the individual ingests the inducing agent to turn on expression of the transgene the inducing agent dose not have any severe toxic side effects on the individual.

The agents that induce an inducible promoter preferably affect only the expression of the gene of interest. Thus, in a bacterium that includes a transgene operably linked to an inducible promoter, the inducing agent does not have any significant affect on the expression of any other genes in the individual.

The agents that induce an inducible promoter preferably are easy to apply or removal. Thus, in a bacterium that includes a transgene operably linked to an inducible promoter that is living in the gut of an individual, the inducing agent is preferably an agent that can be easily delivered to the gut and that can be removed, either by affirmative neutralization for example or by metabolism/passing such that gene expression can be controlled

The agents that induce an inducible promoter preferably induce a clearly detectable expression pattern of either high or very low gene expression.

In some preferred embodiments, the chemically-regulated promoters are derived from organisms distant in evolution to the organisms where its action is required. Examples of inducible or chemically-regulated promoters include tetracycline-regulated promoters. Tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. The Tetracycline resistance operon is carried by the Escherichia coli transposon (Tn) 10. This operon has a negative mode of operation. The interaction between a repressor protein encoded by the operon, TetR, and a DNA sequence to which it binds, the tet operator (tetO), represses the activity of a promoter placed near the operator. In the absence of an inducer, TetR binds to tetO and prevents transcription. Transcription can be turned on when an inducer, such as tetracycline, binds to TetR and causes a conformation change that prevents TetR from remaining bound to the operator. When the operator site is not bound, the activity of the promoter is restored. Tetracycline, the antibiotic, has been used to create two beneficial enhancements to inducible promoters. One enhancement is an inducible on or off promoter. The investigators can choose to have the promoter always activated until Tet is added or always inactivated until Tet is added. This is the Tet on/off promoter. The second enhancement is the ability to regulate the strength of the promoter. The more Tet added, the stronger the effect.

Examples of inducible or chemically-regulated promoters include Steroid-regulated promoters. Steroid-responsive promoters are provided for the modulation of gene expression include promoters based on the rat glucocorticoid receptor (GR); human estrogen receptor (ER); ecdysone receptors derived from different moth species; and promoters from the steroid/retinoid/thyroid receptor superfamily. The hormone binding domain (HBD) of GR and other steroid receptors can also be used to regulate heterologous proteins in cis, that is, operatively linked to protein-encoding sequences upon which it acts. Thus, the HBD of GR, estrogen receptor (ER) and an insect ecdysone receptor have shown relatively tight control and high inducibility

Examples of inducible or chemically-regulated promoters include metal-regulated promoters. Promoters derived from metallothionein (proteins that bind and sequester metal ions) genes from yeast, mouse and human are examples of promoters in which the presence of metals induces gene expression.

IPTG is a classic example of a compound added to cells to activate a promoter. IPTG can be added to the cells to activate the downstream gene or removed to inactivate the gene.

U.S. Pat. No. 6,180,391, which is incorporated herein by reference, refers to the a copper-inducible promoter.

U.S. Pat. No. 6,943,028, which is incorporated herein by reference, refers to highly efficient controlled expression of exogenous genes in E. coli.

U.S. Pat. No. 6,180,367, which is incorporated herein by reference, refers to a process for bacterial production of polypeptides.

Other examples of inducible promoters suitable for use with bacterial hosts include the beta.-lactamase and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P.sub.LtetO-1 and P.sub.lac/are-1 promoters (Lutz and Bujard, Nucleic Acids Res., 25: 1203-1210 (1997), which is incorporated herein by reference), and hybrid promoters such as the tac promoter. deBoer et al., Proc. Nati. Acad. Sci. USA, 80: 21-25 (1983), which is incorporated herein by reference. However, other known bacterial inducible promoters and low-basal-expression promoters are suitable.

U.S. Pat. No. 6,083,715, which is incorporated herein by reference, refers to methods for producing heterologous disulfide bond-containing polypeptides in bacterial cells.

U.S. Pat. No. 5,830,720, which is incorporated herein by reference, refers to recombinant DNA and expression vector for the repressible and inducible expression of foreign genes.

U.S. Pat. No. 5,789,199, which is incorporated herein by reference, refers to a process for bacterial production of polypeptides.

U.S. Pat. No. 5,085,588, which is incorporated herein by reference, refers to bacterial promoters inducible by plant extracts.

U.S. Pat. No. 6,242,194, which is incorporated herein by reference, refers to probiotic bacteria host cells that contain a DNA of interest operably associated with a promoter of the invention can be orally administered to a subject . . . .

U.S. Pat. No. 5,364,780, which is incorporated herein by reference, refers to external regulation of gene expression by inducible promoters.

U.S. Pat. No. 5,639,635, which is incorporated herein by reference, refers to a process for bacterial production of polypeptides.

U.S. Pat. No. 5,789,199, which is incorporated herein by reference, refers to a process for bacterial production of polypeptides.

U.S. Pat. No. 5,689,044, which is incorporated herein by reference, refers to chemically inducible promoter of a plant PR-1 gene.

U.S. Pat. No. 5,063,154, which is incorporated herein by reference, refers to a pheromone-inducible yeast promoter.

U.S. Pat. No. 5,658,565, which is incorporated herein by reference, refers to an inducible nitric oxide synthase gene.

U.S. Pat. Nos. 5,589,392, 6,002,069, 5,693,531, 5,480,794, 6,171,816 6,541,224, 6,495,318, 5,498,538, 5,747,281, 6,635,482 and 5,364,780, which are each incorporated herein by reference, each refer to an IPTG-inducible promoters.

U.S. Pat. Nos. 6,420,170, 5,654,168, 5,912,411, 5,891,718, 6,133,027, 5,739,018, 6,136,954, 6,258,595, 6,002,069 and 6,025,543, which are each incorporated herein by reference, each refer to an tetracycline-inducible promoters.

### Guanylyl Cyclase A (GCA) Agonists (ANP, BNP)

Guanylyl cyclase-A/natriuretic peptide receptor-A (GCA) is a cellular protein involved in maintaining renal and cardiovascular homeostasis. GCA is a receptor found in kidney cells that binds to and is activated by two peptide made in the heart. Atrial natriuretic peptide (ANP, also referred to as cardiac atrial natriuretic peptide) is stored in the heart as pro-ANP and when released, is processed into mature ANP. B-type natriuretic peptide (BNP, also referred to as brain natriuretic peptide) is also produced in the heart. when ANP or BNP bounds to GCA, the GCA-expressing cells produce cGMP as a second messenger. Thus, ANP and BNP are GCA agonistis which activate GCA and lead to accumulation of cGMP in cells expressing GCA.

ANP analogs that are GCA agonists are disclosed in Schiller P W, et al. Superactive analogs of the atrial natriuretic peptide (ANP), Biochem Biophys Res Commun. 1987 Mar. 13; 143(2):499-505; Schiller P W, et al. Synthesis and activity profiles of atrial natriuretic peptide (ANP) analogs with reduced ring size. Biochem Biophys Res Commun. 1986 Jul. 31; 138(2):880-6; Goghari M H, et al. Synthesis and biological activity profiles of atrial natriuretic factor (ANF) analogs., Int J Pept Protein Res. 1990 August; 36(2):156-60; Bovy P R, et al. A synthetic linear decapeptide binds to the atrial natriuretic peptide receptors and demonstrates cyclase activation and vasorelaxant activity. J Biol Chem. 1989 Dec. 5; 264(34):20309-13, and Schoenfeld et al. Molecular Pharmacology January 1995 vol. 47 no. 1 172-180.

### Guanylyl Cyclase B (GCB) Agonists (CNP)

Guanylyl cyclase B (GCB) is also referred to as natriuretic peptide receptor B, atrionatriuretic peptide receptor B and NPR2. GCB is the receptor for a small peptide (C-type natriuretic peptide) produced locally in many different tissues. GCA expression is reported in the kidney, ovarian cells, aorta, chondrocytes, the corpus cavernosum, the pineal gland among other.

While GCB is reported to bind to and be activated by ANP and BNP, C-type natriuretic peptide (CNP) is the most potent activator of GCB. ANP, BNP and CNP are GCB agonists. U.S. Pat. No. 5,434,133 and Furuya, M et al. Biochemical and Biophysical Research Communications, Volume 183, Issue 3, 31 Mar. 1992, Pages 964-969, disclose CNP analogs.

### Soluble Guanylyl Cyclase Activators (Nitric Oxide, Nitrovasodilators, Protoprophyrin IX, and Direct Activators)

Soluble guanylyl cyclase (sGC) is heterodimeric protein made up of an alpha domain with C terminal region that has cyclase activity and a heme-binding beta domain which also has with a C terminal region that has cyclase activity. The sGC which is the only known receptor for nitric oxide has one heme per dimmer. The heme moiety in Fe(II) form is the target of NO. NO bindingresults in activation of sGC, i.e. a substantial increase in sGC activity. Activation of sGC is involved in vasodilation.

YC-1, which is 5-[1-(phenylmethyl)-1H-indazol-3-yl]-2-furanmethanol, is an nitric oxide (NO)-independent activator of soluble guanylyl cyclase. Ko F N et al. YC-1, a novel activator of platelet guanylate cyclase. Blood. 1994 Dec. 15; 84(12):4226-33.

Two drugs that activate sGC are cinaciguat (4-({(4-carboxybutyl)[2-(2-1[4-(2-phenylethyl)phenyl]methoxy}phenyl)ethyl]amino}methyl)benzoic acid) WO-0119780 7,087,644, 7,517,896 WO 20008003414 WO 2008148474 and riociguat, (Methyl N-[4,6-Diamino-2-[1-[(2-fluorophenyl)methyl]-1H-pyrazolo[3,4-b]pyridin-3-yl]-5-pyrimidinyl]-N-methyl-carbaminate) WO-03095451, which has been granted in the US as US-07173037.

Other examples of sGC activators include 3-(5′-hydroxymethyl-2′-furyl)-1-benzylindazole (YC-1, Wu et al., Blood 84 (1994), 4226; Mulsch et al., Brit. J. Pharmacol. 120 (1997), 681), fatty acids (Goldberg et al, J. Biol. Chem. 252 (1977), 1279), diphenyliodonium hexafluorophosphate (Pettibone et al., Eur. J. Pharmacol. 116 (1985), 307), isoliquiritigenin (Yu et. al., Brit. J. Pharmacol. 114 (1995), 1587) and various substituted pyrazole derivatives (WO 98/16223). In addition, WO 98/16507, WO 98/23619, WO 00/06567, WO 00/06568, WO 00/06569, WO 00/21954 WO 02/42299, WO 02/42300, WO 02/42301, WO 02/42302, WO 02/092596 and WO 03/004503 describe pyrazolopyridine derivatives as stimulators of soluble guanylate cyclase. Also described inter alia therein are pyrazolopyridines having a pyrimidine residue in position 3. Compounds of this type have very high in vitro activity in relation to stimulating soluble guanylate cyclase. However, it has emerged that these compounds have disadvantages in respect of their in vivo properties such as, for example, their behavior in the liver, their pharmacokinetic behavior, their dose-response relation or their metabolic pathway.

Other sGC activators are disclosed in O. V. Evgenov et al., Nature Rev. Drug Disc. 5 (2006), 755; and US Published Patent Application Publication Nos. 20110034450, 20100210643, 20100197680, 20100168240, 20100144864, 20100144675, 20090291993, 20090286882, 20090215843, 20080

### PDE Inhibitors

In some embodiments, the active agent comprises PDE inhibitors including, for example, nonselective phosphodiesterase inhibitors, PDE1 selective inhibitors, PDE2 selective inhibitors, PDE3 selective inhibitors, PDE4 selective inhibitors, PDE5 selective inhibitors, and PDE10 selective inhibitors.

PDE inhibitors are generally discussed in the following references which are each incorporated herein by reference: Uzunov, P. and Weiss, B.: Separation of multiple molecular forms of cyclic adenosine 3′,5′-monophosphate phosphodiesterase in rat cerebellum by polyacrylamide gel electrophoresis. Biochim. Biophys. Acta 284:220-226, 1972; Weiss, B.: Differential activation and inhibition of the multiple forms of cyclic nucleotide phosphodiesterase. Adv. Cycl. Nucl. Res. 5:195-211, 1975; Fertel, R. and Weiss, B.: Properties and drug responsiveness of cyclic nucleotide phosphodiesterases of rat lung. Mol. Pharmacol. 12:678-687, 1976; Weiss, B. and Hait, W. N.: Selective cyclic nucleotide phosphodiesterase inhibitors as potential therapeutic agents. Ann. Rev. Pharmacol. Toxicol. 17:441-477, 1977; Essayan D M. (2001). “Cyclic nucleotide phosphodiesterases.”. J Allergy Clin Immunol. 108 (5): 671-80; Deree J, Martins J O, Melbostad H, Loomis W H, Coimbra R. (2008). “Insights into the Regulation of TNF-α Production in Human Mononuclear Cells: The Effects of Non-Specific Phosphodiesterase Inhibition”. Clinics (Sao Paulo). 63 (3): 321-8; Marques L J, Zheng L, Poulakis N, Guzman J, Costabel U (February 1999). “Pentoxifylline inhibits TNF-alpha production from human alveolar macrophages”. Am. J. Respir. Crit. Care Med. 159 (2): 508-11; Peters-Golden M, Canetti C, Mancuso P, Coffey M J. (2005). “Leukotrienes: underappreciated mediators of innate immune responses”. J Immunol. 174 (2): 589-94; Daly J W, Jacobson K A, Ukena D. (1987). “Adenosine receptors: development of selective agonists and antagonists”. Prog Clin Biol Res. 230 (1): 41-63; MacCorquodale D W. THE SYNTHESIS OF SOME ALKYLXANTHINES. Journal of the American Chemical Society. 1929 July; 51(7):2245-2251; WO/1985/002540; U.S. Pat. No. 4,288,433; Daly J W, Padgett W L, Shamim M T (July 1986). “Analogues of caffeine and theophylline: effect of structural alterations on affinity at adenosine receptors”. Journal of Medicinal Chemistry 29 (7): 1305-8; Daly J W, Jacobson K A, Ukena D (1987). “Adenosine receptors: development of selective agonists and antagonists”. Progress in Clinical and Biological Research 230: 41-63; Choi O H, Shamim M T, Padgett W L, Daly J W (1988). “Caffeine and theophylline analogues: correlation of behavioral effects with activity as adenosine receptor antagonists and as phosphodiesterase inhibitors”. Life Sciences 43 (5): 387-98; Shamim M T, Ukena D, Padgett W L, Daly J W (June 1989). “Effects of 8-phenyl and 8-cycloalkyl substituents on the activity of mono-, di-, and trisubstituted alkylxanthines with substitution at the 1-, 3-, and 7-positions”. Journal of Medicinal Chemistry 32 (6): 1231-7; Daly J W, Hide I, Müller C E, Shamim M (1991). “Caffeine analogs: structure-activity relationships at adenosine receptors”. Pharmacology 42 (6): 309-21; Ukena D, Schudt C, Sybrecht G W (February 1993). “Adenosine receptor-blocking xanthines as inhibitors of phosphodiesterase isozymes”. Biochemical Pharmacology 45 (4): 847-51. doi:10.1016/0006-2952(93)90168-V; Daly J W (July 2000). “Alkylxanthines as research tools”. Journal of the Autonomic Nervous System 81 (1-3): 44-52. doi:10.1016/50165-1838(00)00110-7; Daly J W (August 2007). “Caffeine analogs: biomedical impact”. Cellular and Molecular Life Sciences: CMLS 64 (16): 2153-69; González M P, Terán C, Teijeira M (May 2008). “Search for new antagonist ligands for adenosine receptors from QSAR point of view. How close are we?”. Medicinal Research Reviews 28 (3): 329-71; Baraldi P G, Tabrizi M A, Gessi S, Borea P A (January 2008). “Adenosine receptor antagonists: translating medicinal chemistry and pharmacology into clinical utility”. Chemical Reviews 108 (1): 238-63; de Visser Y P, Walther F J, Laghmani E H, van Wijngaarden S, Nieuwland K, Wagenaar G T. (2008). “Phosphodiesterase-4 inhibition attenuates pulmonary inflammation in neonatal lung injury”. Eur Respir J 31 (3): 633-644; Yu M C, Chen J H, Lai C Y, Han C Y, Ko W C. (2009). “Luteolin, a non-selective competitive inhibitor of phosphodiesterases 1-5, displaced [(3)H]-rolipram from high-affinity rolipram binding sites and reversed xylazine/ketamine-induced anesthesia”. Eur J Pharmacol. 627 (1-3): 269-75; Bobon D, Breulet M, Gerard-Vandenhove M A, Guiot-Goffioul F, Plomteux G, Sastre-y-Hernandez M, Schratzer M, Troisfontaines B, von Frenckell R, Wachtel H. (1988). “Is phosphodiesterase inhibition a new mechanism of antidepressant action? A double blind double-dummy study between rolipram and desipramine in hospitalized major and/or endogenous depressives”. Eur Arch Psychiatry Neurol Sci. 238 (1): 2-6; Maxwell C R, Kanes S J, Abel T, Siegel S J. (2004). “Phosphodiesterase inhibitors: a novel mechanism for receptor-independent antipsychotic medications”. Neuroscience. 129 (1): 101-7; Kanes S J, Tokarczyk J, Siegel S J, Bilker W, Abel T, Kelly M P. (2006). “Rolipram: A specific phosphodiesterase 4 inhibitor with potential antipsychotic activity”. Neuroscience. 144 (1): 239-46; and Vecsey C G, Baillie G S, Jaganath D, Havekes R, Daniels A, Wimmer M, Huang T, Brown K M, Li X Y, Descalzi G, Kim S S, Chen T, Shang Y Z, Zhuo M, Houslay M D, Abel T. (2009). “Sleep deprivation impairs cAMP signaling in the hippocampus”. Nature. 461 (7267): 1122-1125.

In addition to activation of guanylyl cyclases, cGMP levels can be elevated and cells protected from chemotherapeutics and radiation therapy using PDE such as PDE 1, PDE2, PDE3, PDE4, PDE5 and PDE10 inhibitors. The breakdown of cGMP is controlled by a family of phosphodiesterase (PDE) isoenzymes. To date, seven members of the family have been described (PDE I-VII) the distribution of which varies from tissue to tissue (Beavo & Reifsnyder (1990) TIPS, 11:150-155 and Nicholson et al (1991) TIPS, 12: 19-27). Specific inhibitors of PDE isoenzymes may be useful to achieve differential elevation of cGMP in different tissues. Some PDE inhibitors specifically inhibit breakdown of cGMP while not effecting cAMP. In some embodiments, possible PDE inhibitors may be PDE3 inhibitors, PDE4 inhibitors, PDE5 inhibitors, PDE3/4 inhibitors or PDE3/4/5 inhibitors.

PDE inhibitors which elevate cGMP specifically are disclosed in U.S. Pat. Nos. 6,576,644, 7,384,958, 7,276,504, 7,273,868, 7,220,736, 7,098,209, 7,087,597, 7,060,721, 6,984,641, 6,930,108, 6,911,469, 6,784,179, 6,656,945, 6,642,244, 6,476,021, 6,326,379, 6,316,438, 6,306,870, 6,300,335, 6,218,392, 6,197,768, 6,037,119, 6,025,494, 6,018,046, 5,869,516, 5,869,486, 5,716,993. Other examples include compounds disclosed in WO 96/05176 and 6,087,368, U.S. Pat. Nos. 4,101,548, 4,001,238, 4,001,237, 3,920,636, 4,060,615, 4,209,623, 5,354,571, 3,031,450, 3,322,755, 5,401,774, 5,147,875, 4,885,301, 4,162,316, 4,047,404, 5,614,530, 5,488,055, 4,880,810, 5,439,895, 5,614,627, GB 2 063 249, EP 0 607 439, WO 97/03985, EP 0 395 328, EP 0 428 268, PCT WO 93/12095, WO 93/07149, EP 0 349 239, EP 0 352 960, EP 0 526 004, EP 0 463 756, EP 0 607 439, WO 94/05661, EP 0 351 058, EP 0 347 146, WO 97/03985, WO 97/03675, WO 95/19978, WO 98/08848, WO 98/16521, EP 0 722 943, EP 0 722 937, EP 0 722 944, WO 98/17668, WO 97/24334, WO 98/06722, PCT/JP97/03592, WO 98/23597, WO 94/29277, WO 98/14448, WO 97/03070, WO 98/38168, WO 96/32379, and PCT/GB98/03712. PDE inhibitors may include those disclosed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 and WO9307124, EP0163965, EP0393500, EP0510562, EP0553174, WO9501338 and WO9603399.

Examples of nonselective phosphodiesterase inhibitors include: methylated xanthines and derivatives such as for examples: caffeine, a minor stimulant, aminophylline, IBMX (3-isobutyl-1-methylxanthine), used as investigative tool in pharmacological research, paraxanthine, pentoxifylline, a drug that has the potential to enhance circulation and may have applicability in treatment of diabetes, fibrotic disorders, peripheral nerve damage, and microvascular injuries, theobromine and theophylline, a bronchodilator. Methylated xanthines act as both competitive nonselective phosphodiesterase inhibitors which raise intracellular cAMP, activate PKA, inhibit TNF-alpha and leukotriene synthesis, and reduce inflammation and innate immunity and nonselective adenosine receptor antagonists. Different analogues show varying potency at the numerous subtypes, and a wide range of synthetic xanthine derivatives (some nonmethylated) have been developed in the search for compounds with greater selectivity for phosphodiesterase enzyme or adenosine receptor subtypes.

PDE inhibitors include 1-(3-Chlorophenylamino)-4-phenylphthalazine and dipyridamol. Another PDE1 selective inhibitor is, for example, Vinpocetine.

PDE2 selective inhibitors include for example, EHNA (erythro-9-(2-hydroxy-3-nonyl)adenine) and Anagrelide.

PDE3 selective inhibitors include for example, sulmazole, ampozone, cilostamide, carbazeran piroximone, imazodan, siguazodan, adibendan, saterinone, emoradan, revizinone, and enoximone and milrinone. Some are used clinically for short-term treatment of cardiac failure. These drugs mimic sympathetic stimulation and increase cardiac output. PDE3 is sometimes referred to as cGMP-inhibited phosphodiesterase.

Examples of PDE3/4 inhibitors include benafentrine, trequinsin, zardaverine and tolafentrine.

PDE4 selective inhibitors include for example: winlcuder, denbufylline, rolipram, oxagrelate, nirtaquazone, motapizone, lixazinone, indolidan, olprinone, atizoram, dipamfylline, arofylline, filaminast, piclamilast, tibenelast, mopidamol, anagrelide, ibudilast, amrinone, pimobendan, cilostazol, quazinone and N-(3,5-dichloropyrid-4-yl)-3-cyclopropylmethoxy4-difluoromethoxybenzamide. Mesembrine, an alkaloid from the herb Sceletium tortuosum; Rolipram, used as investigative tool in pharmacological research; Ibudilast, a neuroprotective and bronchodilator drug used mainly in the treatment of asthma and stroke (inhibits PDE4 to the greatest extent, but also shows significant inhibition of other PDE subtypes, and so acts as a selective PDE4 inhibitor or a non-selective phosphodiesterase inhibitor, depending on the dose); Piclamilast, a more potent inhibitor than rolipram; Luteolin, supplement extracted from peanuts that also possesses IGF-1 properties; Drotaverine, used to alleviate renal colic pain, also to hasten cervical dilatation in labor, and Roflumilast, indicated for people with severe COPD to prevent symptoms such as coughing and excess mucus from worsening. PDE4 is the major cAMP-metabolizing enzyme found in inflammatory and immune cells. PDE4 inhibitors have proven potential as anti-inflammatory drugs, especially in inflammatory pulmonary diseases such as asthma, COPD, and rhinitis. They suppress the release of cytokines and other inflammatory signals, and inhibit the production of reactive oxygen species. PDE4 inhibitors may have antidepressive effects[26] and have also recently been proposed for use as antipsychotics.

PDE5 selective inhibitors include for example: Sildenafil, tadalafil, vardenafil, vesnarinone, zaprinast lodenafil, mirodenafil, udenafil and avanafil. PDE5, is cGMP-specific is responsible for the degradation of cGMP in the corpus cavernosum (these phosphodiesterase inhibitors are used primarily as remedies for erectile dysfunction, as well as having some other medical applications such as treatment of pulmonary hypertension); Dipyridamole (results in added benefit when given together with NO or statins); and newer and more-selective inhibitors are such as icariin, an active component of Epimedium grandiflorum, and possibly 4-Methylpiperazine and Pyrazolo Pyrimidin-7-1, components of the lichen Xanthoparmelia scabrosa.

PDE10 is selective inhibited by Papaverine, an opium alkaloid. PDE10A is almost exclusively expressed in the striatum and subsequent increase in cAMP and cGMP after PDE10A inhibition (e.g. by papaverine) is “a novel therapeutic avenue in the discovery of antipsychotics”.

Additional PDE inhibitors include those set forth in U.S. Pat. Nos. 8,153,104, 8,133,903, 8,114,419, 8,106,061, 8,084,261, 7,951,397, 7,897,633, 7,807,803, 7,795,378, 7,750,015, 7,737,155, 7,732,162, 7,723,342, 7,718,702, 7,671,070, 7,659,273, 7,605,138, 7,585,847, 7,576,066, 7,569,553, 7,563,790, 7,470,687, 7,396,814, 7,393,825, 7,375,100, 7,363,076, 7,304,086, 7,235,625, 7,153,824, 7,091,207, 7,056,936, 7,037,257, 7,022,709, 7,019,010, 6,992,070, 6,969,719, 6,964,780, 6,875,575, 6,743,799, 6,740,306, 6,716,830, 6,670,394, 6,642,244, 6,610,652, 6,555,547, 6,548,508, 6,541,487, 6,538,005, 6,534,519, 6,534,518, 6,479,505, 6,476,025, 6,436,971, 6,436,944, 6,428,478, 6,423,683, 6,399,579, 6,391,869, 6,380,196, 6,376,485, 6,333,354, 6,306,869, 6,303,789, 6,294,564, 6,288,118, 6,271,228, 6,235,782, 6,235,776, 6,225,315, 6,177,471, 6,143,757, 6,143,746, 6,127,378, 6,103,718, 6,080,790, 6,080,782, 6,077,854, 6,066,649, 6,060,501, 6,043,252, 6,011,037, 5,998,428, 5,962,492, 5,922,557, 5,902,824, 5,891,896, 5,874,437, 5,871,780, 5,866,593, 5,859,034, 5,849,770, 5,798,373, 5,786,354, 5,776,958, 5,712,298, 5,693,659, 5,681,961, 5,674,880, 5,622,977, 5,580,888, 5,491,147, 5,426,119, and 5,294,626, which are each incorporated herein by reference. Additional PDE2 inhibitors include those set forth in U.S. Pat. Nos. 6,555,547, 6,538,029, 6,479,493 and 6,465,494, which are each incorporated herein by reference. Additional PDE3 inhibitors include those set forth in U.S. Pat. Nos. 7,375,100, 7,056,936, 6,897,229, 6,716,871, 6,498,173, and 6,110,471, which are each incorporated herein by reference. Additional PDE4 inhibitors include those set forth in U.S. Pat. Nos. 8,153,646, 8,110,682, 8,030,340, 7,964,615, 7,960,433, 7,951,954, 7,902,224, 7,846,973, 7,759,353, 7,659,273, 7,557,247, 7,550,475, 7,550,464, 7,538,127, 7,517,889, 7,446,129, 7,439,393, 7,402,673, 7,375,100, 7,361,787, 7,253,189, 7,135,600, 7,101,866, 7,060,712, 7,056,936, 7,045,658, 6,953,774, 6,884,802, 6,858,596, 6,787,532, 6,747,043, 6,740,655, 6,713,509, 6,630,483, 6,436,971, 6,288,118, and 5,919,801, which are each incorporated herein by reference. Additional PDE5 inhibitors include those set forth in U.S. Pat. Nos. 7,449,462, 7,375,100, 6,969,507, 6,723,719, 6,677,335, 6,660,756, 6,538,029, 6,479,493, 6,476,078, 6,465,494, 6,451,807, 6,143,757, 6,143,746 and 6,043,252, which are each incorporated herein by reference. Additional PDE10 inhibitors include those set forth in U.S. Pat. No. 6,538,029 which is incorporated herein by reference.

### MRP Inhibitors

The human multidrug resistance proteins MRP4 and MRP5 are organic anion transporters that have the unusual ability to transport cyclic nucleotides including cGMP. Accordingly, cGMP levels may be increased by inhibition of MRP4 and MRP5. Compounds that inhibit MRP4 and MRP5 may include dipyridamole, dilazep, nitrobenzyl mercaptopurine riboside, sildenafil, trequinsin, zaprinast and MK571 (3-[[[3-[(1E)-2-(7-Chloro-2-quinolinyl)ethenyl]phenyl][[3-(dimethylamino)-3-oxopropyl]thio]methyl]thio]propanoic acid). These compounds may be more effective at inhibitting MRP4 than MRP5. Other compounds which may be useful as MRP inhibitors include sulfinpyrazone, zidovudine-monophosphate, genistein, indomethacin, and probenecid.

Cyclic GMP and/or cGMP Analogues

In some embodiments, the active agent comprises cyclic GMP. In some embodiments, the active agent comprises cGMP analogues such as for example 8-bromo-cGMP and 2-chloro-cGMP.

### Controlled Release Formulations

Controlled release compositions are provided for delivering to tissues of the duodenum, small intestine, large intestine, colon and/or rectum. The controlled release formulations comprise one or more active agents selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), Guanylyl cyclase C agonists, PDE Inhibitors, MRP inhibitors, cyclic GMP and cGMP analogues, wherein the active agents are formulated as a controlled release composition for controlled release to tissues of the duodenum, small intestine, large intestine, colon and/or rectum.

Methods of preventing or reducing the severity of a disease or condition which is caused or exacerbated by intestinal hyperpermeabilization in an individual identified as being at risk of a disease or condition which is caused or exacerbated by intestinal hyperpermeabilization are provided which comprise the step of administering to the individual by oral administration an amount of the controlled release composition that elevates intracellular cGMP levels in intestinal cells sufficient prevent or reduce intestinal hyperpermeabilization by an amount sufficient to prevent or reduce the severity of the disease or condition which is caused exacerbated by intestinal hyperpermeabilization. In some embodiments, the individual has been identified as being at risk of a disease or disorder selected from the group consisting of: gastrointestinal diseases, dermatologic disorders, hepatobiliary disorders, cardiovascular disorders, pulmonary disorders, autoimmune and collagen vascular disorders, active pulmonary sarcoidosis; neuropsychiatric disorders, local and systemic neoplasms, environmental exposures, genetic disorders and systemic hypersensitivity. Methods of treating an individual who has been identified as having a disease or condition which is caused or exacerbated by intestinal hyperpermeabilization are provided which comprise the step of administering to the individual by oral administration an amount of the controlled release composition that elevates intracellular cGMP levels in intestinal cells sufficient to prevent or reduce intestinal hyperpermeabilization by an amount sufficient to prevent or reduce the severity of the disease or condition which is caused exacerbated by intestinal hyperpermeabilization. In some embodiments, the individual has been identified as having a disease or disorder selected from the group consisting of: gastrointestinal diseases, dermatologic disorders, hepatobiliary disorders, cardiovascular disorders, pulmonary disorders, autoimmune and collagen vascular disorders, active pulmonary sarcoidosis; neuropsychiatric disorders, local and systemic neoplasms, environmental exposures, genetic disorders and systemic hypersensitivity. In methods of preventing and treating individuals: examples of gastrointestinal disease include irritable bowel syndrome, Crohn's disease, ulcerative colitis, and celiac disease; examples of dermatologic disorder include eczema, urticaria-angiedema, psoriasis and dermatitis herpetiformis; examples of hepatobiliary disorder include alcoholic and nonalcoholic liver disease, obstructive jaundice, extrahepatic cholestasis and chronic hepatitis; examples of cardiovascular disorder include chronic heart failure; examples of pulmonary disorder include lung injury induced by ischemia/reperfusion pulmonary hypertension, and hyperoxic lung injury; examples of autoimmune and collagen vascular disorder include vasculitis, systemic sclerosis, Behçet's syndrome, systemic lupus erythematosus, ankylosing spondylitis, postdysenteric reactive arthritis and juvenile idiopathic arthritis; examples neuropsychiatric disorder include autism schizophrenia, seizures, migraine, sensory neuropathy, myasthenia gravis, cerebral vasculitis, multiple sclerosis, and depression; examples of local or systemic neoplasm include colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, and bone cancer; examples of environmental exposure include exposure to an agent selected from the group consisting of Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals; examples of genetic disorders include 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome; and examples of systemic hypersensitivity include asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, and diabetes mellitus. Other diseases or disorders include acne, allergies, fibromyalgia, chronic fatigue syndrome, halitosis, insomnia, nutritional deficiencies, and AIDS.

In some embodiments, methods comprise delivery of one or more active agents selected from the group consisting of: Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon. Bypassing the stomach and releasing the drug after it has passed through the stomach ensures tissue specific delivery of active agent in effective amounts.

The methods provide more effective delivery of active agents to colorectal track including the duodenum, the small and large intestines and the colon. Formulations are provided to deliver active agent throughout the colorectal track or to specific tissue within in.

Some embodiments utilize GCC Agonists, Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors and/or cyclic GMP and/or cGMP analogues and/or PDE inhibitors formulated from controlled release whereby the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon.

Most enteric coatings are intended to protect contents from stomach acid. Accordingly, they are designed to release active agent upon passing through the stomach. The coatings and encapsulations used herein are provided to release active agents upon passing the colorectal track. This can be accomplished in several ways.

Enteric formulations are described in U.S. Pat. No. 4,601,896, U.S. Pat. No. 4,729,893, U.S. Pat. No. 4,849,227, U.S. Pat. No. 5,271,961, U.S. Pat. No. 5,350,741, and U.S. Pat. No. 5,399,347. Oral and rectal formulations are taught in Remington's Pharmaceutical Sciences, 18th Edition, 1990, Mack Publishing Co., Easton Pa. which is incorporated herein by reference.

According to some embodiments, active agents are coated or encapsulated with a sufficient amount of coating material that the time required for the coating material to dissolve and release the active agents corresponds with the time required for the coated or encapsulated composition to travel from the mouth to the colorectal track.

According to some embodiments, the active agents are coated or encapsulated with coating material that does not fully dissolve and release the active agents until it comes in contact with conditions present in the colorectal track. Such conditions may include the presence of enzymes in the colorectal track, pH, tonicity, or other conditions that vary relative to the small intestine.

According to some embodiments, the active agents are coated or encapsulated with coating material that is designed to dissolve in stages as it passes from stomach to small intestine to large intestine. The active agents are released upon dissolution of the final stage which occurs in the colorectal track.

In some embodiments, the formulations are provided for release of active agent in specific tissues or regions of the colorectal track, for example, the duodenum, the small intestine, the large intestine or the colon.

Examples of technologies which may be used to formulate active agents for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference. Controlled release formulations are well known including those which are particularly suited for release of active agent into the duodenum. Examples of controlled release formulations which may be used include U.S. Patent Application Publication 2010/0278912, U.S. Pat. No. 4,792,452, U.S. Patent Application Publication 2005/0080137, U.S. Patent Application Publication 2006/0159760, U.S. Patent Application Publication 2011/0251231, U.S. Pat. No. 5,443,843, U.S. Patent Application Publication 2008/0153779, U.S. Patent Application Publication 2009/0191282, U.S. Patent Application Publication 2003/0228362, U.S. Patent Application Publication 2004/0224019, U.S. Patent Application Publication 2010/0129442, U.S. Patent Application Publication 2007/0148153, U.S. Pat. No. 5,536,507, U.S. Pat. No. 7,790,755, U.S. Patent Application Publication 2005/0058704, U.S. Patent Application Publication 2001/0026800, U.S. Patent Application Publication 2009/0175939, US 2002/0192285, U.S. Patent Application Publication 2008/0145417, U.S. Patent Application Publication 2009/0053308, U.S. Pat. No. 8,043,630, U.S. Patent Application Publication 2011/0053866, U.S. Patent Application Publication 2009/0142378, U.S. Patent Application Publication 2006/0099256, U.S. Patent Application Publication 2009/0104264, U.S. Patent Application Publication 2004/0052846, U.S. Patent Application Publication 2004/0053817, U.S. Pat. No. 4,013,784, U.S. Pat. No. 5,693,340, U.S. Patent Application Publication 2011/0159093, U.S. Patent Application Publication 2009/0214640, U.S. Pat. No. 5,133,974, U.S. Pat. No. 5,026,559, U.S. Patent Application Publication 2010/0166864, U.S. Patent Application Publication 2002/0110595, U.S. Patent Application Publication 2007/0148153, U.S. Patent Application Publication 2009/0220611, U.S. Patent Application Publication 2010/0255087 and U.S. Patent Application Publication 2009/0042889, each of which is incorporated herein by reference. Other examples of technologies which may be used to formulate active agents for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

### Protection of Intestinal Cells in Patients

Patients may be provided with compositions which elevate cGMP levels in gastrointestinal tissue in order to protect those tissues.

Protection of intestinal cells can be achieved by elevation of cGMP levels. The elevation of cGMP levels in intestinal cells may be achieved by administration of one or more compounds in amounts sufficient to achieve elevated cGMP levels. The one or more compounds are delivered to intestinal cells in amounts and frequency sufficient to sustain the cGMP at elevated levels.

In some embodiments, compounds which elevate cGMP do so through interaction with a cellular receptor present on the cells. GCC agonists may be delivered by routes that provide the agonist to contact the GCC expressed by intestinal cells in order to activate the receptors. In some embodiments, the compounds which elevate cGMP levels may be taken up by cell by other means. For example, cells which contain specific PDE or MRP isoforms would indicate the inhibitory compounds used. For example, cells expressing PDE5 would be protected by use of PDE5 inhibitors while cells expressing MRP5 would be protected by use of MRP5 inhibitors. In such embodiments, the compounds may be administered by any route such that they can be taken up by cells.

In embodiments in which cGMP levels are to be increased in normal intestinal cells using GCC agonists, oral delivery to the gut is preferred. Compounds must be protected from degradation or uptake prior to reaching the gut. Many known peptide agonists of GCC are stable in the acidic environment of the stomach and will survive in active form when passing through the stomach to the gut. Some compounds may require enteric coating. In the case of GCC expression in cell lining the gut, the delivery of GCC agonist through local delivery directly to the interior of the intestinal, by oral or rectal administration for example, is particularly useful in that cells outside the gut will not be exposed to the GCC agonist since the tight junctions of intestinal tissue prevent direct passage of most GCC agonists.

In some embodiments the one or more compounds which increase cGMP levels is formulated as a injectable pharmaceutical composition suitable for parenteral administration such as by intravenous, intraarterial, intramuscular, intradermal or subcutraneous injection. Accordingly, the composition is a sterile, pyrogen-free preparation that has the structural/physical characteristics required for injectable products; i.e. it meets well known standards recognized by those skilled in the art for purity, pH, isotonicity, sterility, and particulate matter.

In some preferred embodiments, the one or more compounds which increase cGMP levels is administered orally or rectally and the compositions is formulated as pharmaceutical composition suitable for oral or rectal administration. Some embodiments providing the one or more compounds which increase cGMP levels are provided as suitable for oral administration and formulated for sustained release. Some embodiments providing the one or more compounds which increase cGMP levels are provided as suitable for oral administration and formulated by enteric coating to release the active agent in the intestine. Enteric formulations are described in U.S. Pat. No. 4,601,896, U.S. Pat. No. 4,729,893, U.S. Pat. No. 4,849,227, U.S. Pat. No. 5,271,961, U.S. Pat. No. 5,350,741, and U.S. Pat. No. 5,399,347. Oral and rectal formulation are taught in Remington's Pharmaceutical Sciences, 18th Edition, 1990, Mack Publishing Co., Easton Pa. which is incorporated herein by reference.

Alternative embodiments include sustained release formulations and implant devices which provide continuous delivery of. the one or more compounds which increase cGMP levels. In some embodiments, the one or more compounds which increase cGMP levels is administered topically, intrathecally, intraventricularly, intrapleurally, intrabronchially, or intracranially.

Generally, the one or more compounds which increase cGMP levels must be present at a sufficient level for a sustained amount of time to increase cGMP levels. Generally, enough of the one or more compounds which increase cGMP levels must be administered initially and/or by continuous administration to maintain the concentration of sufficient to maintain elevated cGMP levels. It is preferred that elevated cGMP levels sufficient to enhance cell survival and barrier integrity be maintained Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

In some embodiments, a GCC agonist such as a peptide having SEQ ID NO:2, 3 or 5-58 is administered to the individual. In practicing the method, the compounds may be administered singly or in combination with other compounds. In the method, the compounds are preferably administered with a pharmaceutically acceptable carrier selected on the basis of the selected route of administration and standard pharmaceutical practice. It is contemplated that the daily dosage of a compound used in the method will be in the range of from about 1 micrograms to about 10 grams per day. In some preferred embodiments, the daily dosage compound will be in the range of from about 10 mg to about 1 gram per day. In some preferred embodiments, the daily dosage compound will be in the range of from about 100 mg to about 500 mg per day. It is contemplated that the daily dosage of a compound used in the method that is the invention will be in the range of from about 1 μg to about 100 mg per kg of body weight, in some embodiments, from about 1 μg to about 40 mg per kg body weight; in some embodiments from about 10 μg to about 20 mg per kg per day, and in some embodiments 10 μg to about 1 mg per kg per day. Pharmaceutical compositions may be administered in a single dosage, divided dosages or in sustained release. In some preferred embodiments, the compound will be administered in multiple doses per day. In some preferred embodiments, the compound will be administered in 3-4 doses per day. The method of administering compounds include administration as a pharmaceutical composition orally in solid dosage forms, such as capsules, tablets, and powders, or in liquid dosage forms, such as elixirs, syrups, and suspensions. Compounds may be mixed with powdered carriers, such as lactose, sucrose, mannitol, starch, cellulose derivatives, magnesium stearate, and stearic acid for insertion into gelatin capsules, or for forming into tablets. Both tablets and capsules may be manufactured as sustained release products for continuous release of medication over a period of hours. Compressed tablets can be sugar or film coated to mask any unpleasant taste and protect the tablet from the atmosphere or enteric coated for selective disintegration in the gastrointestinal tract. In some preferred embodiments, compounds are delivered orally and are coated with an enteric coating which makes the compounds available upon passing through the stomach and entering the intestinal tract, preferably upon entering the large intestine. U.S. Pat. No. 4,079,125, which is incorporated herein by reference, teaches enteric coating which may be used to prepare enteric coated compound of the inventions useful in the methods of the invention. Liquid dosage forms for oral administration may contain coloring and flavoring to increase patient acceptance, in addition to a pharmaceutically acceptable diluent such as water, buffer or saline solution. For parenteral administration, a compound may be mixed with a suitable carrier or diluent such as water, a oil, saline solution, aqueous dextrose (glucose), and related sugar solutions, and glycols such as propylene glycol or polyethylene glycols. Solutions for parenteral administration contain preferably a water soluble salt of the compound. Stabilizing agents, antioxidizing agents and preservatives may also be added. Suitable antioxidizing agents include sodium bisulfite, sodium sulfite, and ascorbic acid, citric acid and its salts, and sodium EDTA. Suitable preservatives include benzalkonium chloride, methyl- or propyl-paraben, and chlorbutanol

### EXAMPLES

### Example 1

We have employed GCC deficient (GCC KO) mice to examine the role of GCC in barrier maintenance. Microarray analysis of normal and GCC KO mice identified tight junctions a potential target for GCC signaling. Electron microscopic evaluation of tight junctions revealed morphologic changes in GCC KO mice. These data were supported by direct measurements of barrier permeability using FITC-dextran. Pathology induced by administration of dextran sulfate sodium (DSS), a chemical model of inflammatory bowel disease, was exacerbated in GCC KO mice resulting in systemic bacterial translocation and 85% mortality. Importantly, GCC deficiency also increased liver tumorigenesis following azoxymethane (AOM) administration, as well as spontaneous liver and lymphocyte tumorigenesis (lymphoma), all of which were absent in normal mice.

### Example 2

Administration of one or more GCC agonists in an amount effective prevent hyperpermeabilization of the intestinal barrier prevents or reduces the diffusion of microorganisms macromolecules, antigens, and metabolic and microbial toxins in the gut into the body outside of the intestinal lumen and in some cases into access the blood stream. A breach of the integrity of the intestine to separate the contents of the gut from the rest of the body and thereby allow only selective entry of materials from the gut compromises the health and well being of the individual. Use of one or more GCC agonists in an amount effective prevent hyperpermeabilization of the intestinal barrier assists in maintaining the health and well being of the individual be preventing or reducing the diseases and condition which occur when the integrity of the barrier is compromised. One or more GCC agonists and/or one or more PDE inhibitors and/or one or more MRP inhibitors may be administered in an amount effective elevate cGMP levels sufficient to prevent or reduce breaches in the intestinal barrier.

In some embodiments, one or more GCC agonists and/or one or more PDE inhibitors and/or one or more MRP inhibitors may be administered to individuals to prevent or reduce breaches in the intestinal barrier and thereby preventing or reducing the subsequent effects of such breach which lead to gastrointestinal diseases, dermatologic disorders, hepatobiliary disorders, cardiovascular disorders, pulmonary disorders, autoimmune and collagen vascular disorders, active pulmonary sarcoidosis; neuropsychiatric disorders, local and systemic neoplasms, environmental exposures, genetic disorders, systemic hypersensitivity, and other potential diseases.

### Example 3

In some embodiments, methods of preventing or reducing gastrointestinal disease, including, but not limited to, irritable bowel syndrome, Crohn's disease, ulcerative colitis, and celiac disease may be performed. In some embodiments, individuals who are treated may be identified as being predisposed or at an elevated risk compared to normal risk to develop gastrointestinal disease, including, but not limited to, irritable bowel syndrome, Crohn's disease, ulcerative colitis, and celiac disease. In some embodiments, individuals who are treated may be first tested to identify them as having early stages, being predisposed or at an elevated risk compared to normal risk to develop gastrointestinal disease, including, but not limited to, irritable bowel syndrome, Crohn's disease, ulcerative colitis, and celiac disease.

In individuals who are identified as being susceptible to gastrointestinal disease, including, but not limited to, irritable bowel syndrome, Crohn's disease, ulcerative colitis, and celiac disease, activation of GCC or other methods of increasing cGMP disclosed herein may be undertaken in amounts sufficient to maintain barrier integrity over long periods of time, i.e. for example chronic treatment as a prophylactic means of maintaining intestinal heath. Likewise, in individuals identified as suffering from gastrointestinal disease, including, but not limited to, irritable bowel syndrome, Crohn's disease, ulcerative colitis, and celiac disease, treatment to activate GCC or otherwise increase cGMP levels may be undertaken to provide remedial or adjunctive therapy by administering in amounts sufficient to restore barrier integrity.

### Example 4

In some embodiments, methods of preventing or reducing dermatologic disorders, including, but not limited to, eczema, urticaria-angiedema, psoriasis and dermatitis herpetiformis may be performed. In some embodiments, individuals who are treated may be identified as being predisposed or at an elevated risk compared to normal risk to develop dermatologic disorders, including, but not limited to, eczema, urticaria-angiedema, psoriasis and dermatitis herpetiformis. In some embodiments, individuals who are treated may be first tested to identify them as having early stages, being predisposed or at an elevated risk compared to normal risk to develop dermatologic disorders, including, but not limited to, eczema, urticaria-angiedema, psoriasis and dermatitis herpetiformis.

In individuals who are identified as being susceptible to dermatologic disorders, including, but not limited to, eczema, urticaria-angiedema, psoriasis and dermatitis herpetiformis, activation of GCC or other methods of increasing cGMP disclosed herein may be undertaken in amounts sufficient to maintain barrier integrity over long periods of time, i.e. for example chronic treatment as a prophylactic means of maintaining intestinal heath sufficient to prevent or reduce dermatologic disorders, including, but not limited to, eczema, urticaria-angiedema, psoriasis and dermatitis herpetiformis. Likewise, in individuals identified as suffering from dermatologic disorders, including, but not limited to, eczema, urticaria-angiedema, psoriasis and dermatitis herpetiformis, treatment to activate GCC or otherwise increase cGMP levels may be undertaken to provide remedial or adjunctive therapy by administering in amounts sufficient to restore barrier integrity such that the dermatologic disorders, including, but not limited to, eczema, urticaria-angiedema, psoriasis and dermatitis herpetiformis are resolved or reduced.

### Example 5

In some embodiments, methods of preventing or reducing hepatobiliary disorders, including, but not limited to, alcoholic and nonalcoholic liver disease, obstructive jaundice, extrahepatic cholestasis, chronic hepatitis may be performed. In some embodiments, individuals who are treated may be identified as being predisposed or at an elevated risk compared to normal risk to develop hepatobiliary disorders, including, but not limited to, alcoholic and nonalcoholic liver disease, obstructive jaundice, extrahepatic cholestasis, chronic hepatitis. In some embodiments, individuals who are treated may be first tested to identify them as having early stages, being predisposed or at an elevated risk compared to normal risk to develop hepatobiliary disorders, including, but not limited to, alcoholic and nonalcoholic liver disease, obstructive jaundice, extrahepatic cholestasis, chronic hepatitis.

In individuals who are identified as being susceptible to hepatobiliary disorders, including, but not limited to, alcoholic and nonalcoholic liver disease, obstructive jaundice, extrahepatic cholestasis, chronic hepatitis, activation of GCC or other methods of increasing cGMP disclosed herein may be undertaken in amounts sufficient to maintain barrier integrity over long periods of time, i.e. for example chronic treatment as a prophylactic means of maintaining intestinal heath sufficient to prevent or reduce hepatobiliary disorders, including, but not limited to, alcoholic and nonalcoholic liver disease, obstructive jaundice, extrahepatic cholestasis, chronic hepatitis. Likewise, in individuals identified as suffering from hepatobiliary disorders, including, but not limited to, alcoholic and nonalcoholic liver disease, obstructive jaundice, extrahepatic cholestasis, chronic hepatitis symptoms, treatment to activate GCC or otherwise increase cGMP levels may be undertaken to provide remedial or adjunctive therapy by administering in amounts sufficient to restore barrier integrity such that the hepatobiliary disorders, including, but not limited to, alcoholic and nonalcoholic liver disease, obstructive jaundice, extrahepatic cholestasis, chronic hepatitis symptoms are resolved or reduced.

### Example 6

In some embodiments, methods of preventing or reducing preventing or reducing cardiovascular disorders, including, but not limited to, chronic heart failure (CHF), may be performed. In some embodiments, individuals who are treated may be identified as being predisposed or at an elevated risk compared to normal risk to develop preventing or reducing cardiovascular disorders, including, but not limited to, chronic heart failure (CHF). In some embodiments, individuals who are treated may be first tested to identify them as having early stages, being predisposed or at an elevated risk compared to normal risk to develop preventing or reducing cardiovascular disorders, including, but not limited to, chronic heart failure (CHF).

In individuals who are identified as being susceptible to h cardiovascular disorders, including, but not limited to, chronic heart failure (CHF), activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce cardiovascular disorders, including, but not limited to, chronic heart failure (CHF). Likewise, in individuals identified as suffering from cardiovascular disorders, including, but not limited to, chronic heart failure (CHF), treatment to activate GCC or otherwise increase cGMP levels may be undertaken to provide remedial or adjunctive therapy by administering in amounts sufficient to restore barrier integrity such that the cardiovascular disorders, including, but not limited to, chronic heart failure (CHF) are resolved or reduced.

### Example 7

In some embodiments, methods of preventing or reducing pulmonary disorders, including, but not limited to, lung injury induced by ischemia/reperfusion pulmonary hypertension, or hyperoxic lung injury may be performed. In some embodiments, individuals who are treated may be identified as being predisposed or at an elevated risk compared to normal risk to develop pulmonary disorders, including, but not limited to, lung injury induced by ischemia/reperfusion pulmonary hypertension, or hyperoxic lung injury. In some embodiments, individuals who are treated may be first tested to identify them as having early stages, being predisposed or at an elevated risk compared to normal risk to develop pulmonary disorders, including, but not limited to, lung injury induced by ischemia/reperfusion pulmonary hypertension, or hyperoxic lung injury.

In individuals who are identified as being susceptible to pulmonary disorders, including, but not limited to, lung injury induced by ischemia/reperfusion pulmonary hypertension, or hyperoxic lung injury, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to pulmonary disorders, including, but not limited to, lung injury induced by ischemia/reperfusion pulmonary hypertension, or hyperoxic lung injury. Likewise, in individuals identified as suffering from pulmonary disorders, including, but not limited to, lung injury induced by ischemia/reperfusion pulmonary hypertension, or hyperoxic lung injury, treatment to activate GCC or otherwise increase cGMP levels may be undertaken to provide remedial or adjunctive therapy by administering in amounts sufficient to restore barrier integrity such that the pulmonary disorders, including, but not limited to, lung injury induced by ischemia/reperfusion pulmonary hypertension, or hyperoxic lung injury are resolved or reduced.

### Example 8

In some embodiments, methods of preventing or reducing autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis may be performed. In some embodiments, individuals who are treated may be identified as being predisposed or at an elevated risk compared to normal risk to develop autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis. In some embodiments, individuals who are treated may be first tested to identify them as having early stages, being predisposed or at an elevated risk compared to normal risk to develop autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis.

In individuals who are identified as being susceptible to autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis. Likewise, in individuals identified as suffering from autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis, treatment to activate GCC or otherwise increase cGMP levels may be undertaken to provide remedial or adjunctive therapy by administering in amounts sufficient to restore barrier integrity such that the autoimmune and collagen vascular disorders, including, but not limited to, vasculitis, systemic sclerosis, Behçet's syndrome, systemic lupus erythematosus, ankylosing spondylitis, postdysenteric reactive arthritis and juvenile idiopathic arthritis are resolved or reduced.

### Example 9

In some embodiments, methods of preventing or reducing active pulmonary sarcoidosis may be performed. In some embodiments, individuals who are treated may be identified as being predisposed or at an elevated risk compared to normal risk to develop active pulmonary sarcoidosis. In some embodiments, individuals who are treated may be first tested to identify them as having early stages, being predisposed or at an elevated risk compared to normal risk to develop active pulmonary sarcoidosis.

In individuals who are identified as being susceptible to active pulmonary sarcoidosis, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce severity of active pulmonary sarcoidosis. Likewise, in individuals identified as suffering from active pulmonary sarcoidosis, treatment to activate GCC or otherwise increase cGMP levels may be undertaken to provide remedial or adjunctive therapy by administering in amounts sufficient to restore barrier integrity such that the active pulmonary sarcoidosis symptoms are resolved or reduced.

### Example 10

In some embodiments, methods of preventing or reducing neuropsychiatric disorders, including, but not limited to, autism schizophrenia, seizures, migraine, sensory neuropathy, myasthenia gravis, cerebral vasculitis, multiple sclerosis, and depression may be performed. In some embodiments, individuals who are treated may be identified as being predisposed or at an elevated risk compared to normal risk to develop neuropsychiatric disorders, including, but not limited to, autism schizophrenia, seizures, migraine, sensory neuropathy, myasthenia gravis, cerebral vasculitis, multiple sclerosis, and depression. In some embodiments, individuals who are treated may be first tested to identify them as having early stages, being predisposed or at an elevated risk compared to normal risk to develop neuropsychiatric disorders, including, but not limited to, autism schizophrenia, seizures, migraine, sensory neuropathy, myasthenia gravis, cerebral vasculitis, multiple sclerosis, and depression.

In individuals who are identified as being susceptible to neuropsychiatric disorders, including, but not limited to, autism schizophrenia, seizures, migraine, sensory neuropathy, myasthenia gravis, cerebral vasculitis, multiple sclerosis, and depression activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce severity of neuropsychiatric disorders, including, but not limited to, autism schizophrenia, seizures, migraine, sensory neuropathy, myasthenia gravis, cerebral vasculitis, multiple sclerosis, and depression. Likewise, in individuals identified as suffering from active neuropsychiatric disorders, including, but not limited to, autism schizophrenia, seizures, migraine, sensory neuropathy, myasthenia gravis, cerebral vasculitis, multiple sclerosis, and depression, treatment to activate GCC or otherwise increase cGMP levels may be undertaken to provide remedial or adjunctive therapy by administering in amounts sufficient to restore barrier integrity such that the neuropsychiatric disorders, including, but not limited to, autism schizophrenia, seizures, migraine, sensory neuropathy, myasthenia gravis, cerebral vasculitis, multiple sclerosis, and depression symptoms are resolved or reduced.

### Example 11

In some embodiments, methods of preventing or reducing local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer may be performed. In some embodiments, individuals who are treated may be identified as being predisposed or at an elevated risk compared to normal risk to develop local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer. In some embodiments, individuals who are treated may be first tested to identify them as having early stages, being predisposed or at an elevated risk compared to normal risk to develop local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer.

In individuals who are identified as being susceptible to local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, and bone cancer, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce severity of local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer. Likewise, in individuals identified as suffering from local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, bone cancer, treatment to activate GCC or otherwise increase cGMP levels may be undertaken to provide remedial or adjunctive therapy by administering in amounts sufficient to restore barrier integrity such that the local and systemic neoplasms, including colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, and bone cancer is reduced or in remission.

### Example 12

In some embodiments, methods of preventing or reducing severity of health consequences associated with Environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals may be performed. In some embodiments, individuals who are treated may be identified as being predisposed or at an elevated risk compared to normal risk to develop severity of health consequences associated with Environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals. In some embodiments, individuals who are treated may be first tested to identify them as having early stages, being predisposed or at an elevated risk compared to normal risk to develop severity of health consequences associated with Environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals.

In individuals who are identified as being susceptible to health consequences associated with environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce severity of health consequences associated with environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals. Likewise, in individuals identified as suffering from health consequences associated with Environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals, treatment to activate GCC or otherwise increase cGMP levels may be undertaken to provide remedial or adjunctive therapy by administering in amounts sufficient to restore barrier integrity such that the severity of health consequences associated with environmental exposures, including but not limited to, Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals is reduced.

### Example 13

In some embodiments, methods of preventing or reducing severity of health of consequences due to genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome may be performed. In some embodiments, individuals who are treated may be identified as being predisposed or at an elevated risk compared to normal risk to develop severity of health of consequences due to Genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome. In some embodiments, individuals who are treated may be first tested to identify them as having early stages, being predisposed or at an elevated risk compared to normal risk to develop severity of health of consequences due to Genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome.

In individuals who are identified as being susceptible to health consequences associated with due to genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce severity of health of consequences due to genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome. Likewise, in individuals identified as suffering from health of consequences due to genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome, treatment to activate GCC or otherwise increase cGMP levels may be undertaken to provide remedial or adjunctive therapy by administering in amounts sufficient to restore barrier integrity such that the severity of health of consequences due to genetic disorders, including but not limited to, 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome is reduced.

### Example 14

In some embodiments, methods of preventing or reducing systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus may be performed. In some embodiments, individuals who are treated may be identified as being predisposed or at an elevated risk compared to normal risk to develop systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus. In some embodiments, individuals who are treated may be first tested to identify them as having early stages, being predisposed or at an elevated risk compared to normal risk to develop systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus.

In individuals who are identified as being susceptible to systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus. Likewise, in individuals identified as suffering from systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus, treatment to activate GCC or otherwise increase cGMP levels may be undertaken to provide remedial or adjunctive therapy by administering in amounts sufficient to restore barrier integrity such that systemic hypersensitivity, including, but not limited to, asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, diabetes mellitus is resolved or reduced.

### Example 15

Thus in some embodiments, methods of preventing or reducing other potential diseases including, but not limited to, acne, allergies, fibromyalgia, chronic fatigue syndrome, halitosis, insomnia, nutritional deficiencies, and AIDS may be performed. In some embodiments, individuals who are treated may be identified as being predisposed or at an elevated risk compared to normal risk to develop other potential diseases including, but not limited to, acne, allergies, fibromyalgia, chronic fatigue syndrome, halitosis, insomnia, nutritional deficiencies, and AIDS. In some embodiments, individuals who are treated may be first tested to identify them as having early stages, being predisposed or at an elevated risk compared to normal risk to develop other potential diseases including, but not limited to, acne, allergies, fibromyalgia, chronic fatigue syndrome, halitosis, insomnia, nutritional deficiencies, and AIDS.

In individuals who are identified as being susceptible to other potential diseases including, but not limited to, acne, allergies, fibromyalgia, chronic fatigue syndrome, halitosis, insomnia, nutritional deficiencies, and AIDS, activation of GCC or other methods of increasing cGMP disclosed herein may be undertaken in amounts sufficient to maintain barrier integrity over long periods of time, i.e. for example chronic use of treatment as a prophylactic means of maintaining intestinal heath sufficient to prevent or reduce other potential diseases including, but not limited to, acne, allergies, fibromyalgia, chronic fatigue syndrome, halitosis, insomnia, deficiencies, and AIDS. Likewise, in individuals identified as suffering from other potential diseases including, but not limited to, acne, allergies, fibromyalgia, chronic fatigue syndrome, halitosis, insomnia, nutritional deficiencies, and AIDS, treatment to activate GCC or otherwise increase cGMP levels may be undertaken to provide remedial or adjunctive therapy by administering in amounts sufficient to restore barrier integrity such that the severity other potential diseases including, but not limited to, acne, allergies, fibromyalgia, chronic fatigue syndrome, halitosis, insomnia, nutritional deficiencies, and AIDS is reduced.

1. A method of preventing or reducing the severity of a disease or condition which is caused or exacerbated by intestinal hyperpermeabilization in an individual identified as being at risk of a disease or condition which is caused or exacerbated by intestinal hyperpermeabilization comprising the step of:
administering to the individual an amount of one or more compounds that elevates intracellular cGMP levels in intestinal cells sufficient prevent or reduce intestinal hyperpermeabilization by an amount sufficient to prevent or reduce the severity of the disease or condition which is caused exacerbated by intestinal hyperpermeabilization.

2. The method of claim 1 wherein the individual has been identified as being at risk of a disease or disorder selected from the group consisting of: gastrointestinal diseases, dermatologic disorders, hepatobiliary disorders, cardiovascular disorders, pulmonary disorders, autoimmune and collagen vascular disorders, active pulmonary sarcoidosis; neuropsychiatric disorders, local and systemic neoplasms, environmental exposures, genetic disorders and systemic hypersensitivity.

3. A method of treating an individual who has been identified as having a disease or condition which is caused or exacerbated by intestinal hyperpermeabilization comprising the step of:
administering to the individual an amount of one or more compounds that elevates intracellular cGMP levels in intestinal cells sufficient prevent or reduce intestinal hyperpermeabilization by an amount sufficient to prevent or reduce the severity of the disease or condition which is caused or exacerbated by intestinal hyperpermeabilization.

4. The method of claim 3 wherein the individual has been identified as having a disease or disorder selected from the group consisting of: gastrointestinal diseases, dermatologic disorders, hepatobiliary disorders, cardiovascular disorders, pulmonary disorders, autoimmune and collagen vascular disorders, active pulmonary sarcoidosis; neuropsychiatric disorders, local and systemic neoplasms, environmental exposures, genetic disorders and systemic hypersensitivity.

5. The method of claim 2 wherein:
the gastrointestinal disease is selected from the group consisting of irritable bowel syndrome, Crohn's disease, ulcerative colitis, and celiac disease;
the dermatologic disorder is selected from the group consisting of eczema, urticaria-angiedema, psoriasis and dermatitis herpetiformis;
the hepatobiliary disorder is selected from the group consisting of alcoholic and nonalcoholic liver disease, obstructive jaundice, extrahepatic cholestasis and chronic hepatitis;
the cardiovascular disorder is chronic heart failure;
the pulmonary disorder is selected from the group consisting of lung injury induced by ischemia/reperfusion pulmonary hypertension, and hyperoxic lung injury;
the autoimmune and collagen vascular disorder is selected from the group consisting of vasculitis, systemic sclerosis, Behçet's syndrome, systemic lupus erythematosus, ankylosing spondylitis, postdysenteric reactive arthritis and juvenile idiopathic arthritis;
the disease or disorder is pulmonary sarcoidosis;
the neuropsychiatric disorder is selected from the group consisting of autism schizophrenia, seizures, migraine, sensory neuropathy, myasthenia gravis, cerebral vasculitis, multiple sclerosis, and depression;
the local or systemic neoplasm is selected from the group consisting of colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, and bone cancer;
the environmental exposure is exposure to an agent selected from the group consisting of Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals;
the genetic disorder is selected from the group consisting of 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome;
the systemic hypersensitivity is selected from the group consisting of asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, and diabetes mellitus; and
the disease or disorder is selected from the group consisting of acne, allergies, fibromyalgia, chronic fatigue syndrome, halitosis, insomnia, nutritional deficiencies, and AIDS.

6. The method of claim 4 wherein
the gastrointestinal disease is selected from the group consisting of irritable bowel syndrome, Crohn's disease, ulcerative colitis, and celiac disease;
the dermatologic disorder is selected from the group consisting of eczema, urticaria-angiedema, psoriasis and dermatitis herpetiformis;
the hepatobiliary disorder is selected from the group consisting of alcoholic and nonalcoholic liver disease, obstructive jaundice, extrahepatic cholestasis and chronic hepatitis;
the cardiovascular disorder is chronic heart failure;
the pulmonary disorder is selected from the group consisting of lung injury induced by ischemia/reperfusion pulmonary hypertension, and hyperoxic lung injury;
the autoimmune and collagen vascular disorder is selected from the group consisting of vasculitis, systemic sclerosis, Behçet's syndrome, systemic lupus erythematosus, ankylosing spondylitis, postdysenteric reactive arthritis and juvenile idiopathic arthritis;
the disease or disorder is pulmonary sarcoidosis;
the neuropsychiatric disorder is selected from the group consisting of autism schizophrenia, seizures, migraine, sensory neuropathy, myasthenia gravis, cerebral vasculitis, multiple sclerosis, and depression;
the local or systemic neoplasm is selected from the group consisting of colorectal cancer, hepatocellular carcinoma, breast cancer, leukemias, lymphomas, lung cancers, prostate cancer, pancreatic cancer, gastric cancer, esophageal cancer, ovarian cancer, MALT and GALT lymphoma, throat cancer, ovary cancer, uterine corpus and cervical cancer, renal cell carcinomas, bladder cancer, and bone cancer;
the environmental exposure is exposure to an agent selected from the group consisting of Styrene, 1,4-Dichlorobenzene, Xylene, Ethylphenol, OCDD (dioxin), HxCDD (dioxin), 1,2,3,4,7,8,9-HpCDD, Benzene, Chlorobenzene, Ethylbenzene, p,p′-DDE, 1,2,3,4,6,7,8-HpCDF, 1,2,3,7,8,-PeCDD, Toluene, 2,3,4,7,8-PeCDF, Beta-BHC, Total PCBs, Chloroform, Hexachlorobenzene, 2,3,7,8-TCDD, and other agents such as pesticides, cleaning and manufacturing chemicals;
the genetic disorder is selected from the group consisting of 22q11.2 deletion syndrome, Angelman syndrome, Canavan disease, Celiac disease, Charcot-Marie-Tooth disease, Color blindness, Cri du chat, Cystic fibrosis, Down syndrome, Duchenne muscular dystrophy, Haemophilia, Klinefelter's syndrome, Neurofibromatosis, Phenylketonuria, Prader-Willi syndrome, Sickle-cell disease, Tay-Sachs disease and Turner syndrome;
the systemic hypersensitivity is selected from the group consisting of asthma, food allergy, eczema, Rheumatoid arthritis ITP, hemolytic anemia, pernicious anemia, Still's disease, transfusion reactions due to anti-IgA antibody, dermatomyositis, vitiligo, Sjogren's syndrome, Henoch-Schonlein syndrome, primary biliary cirrhosis, autoimmune hepatitis, thyroiditis, Graves disease, idiopathic Addision's disease, and diabetes mellitus; and
the disease or disorder is selected from the group consisting of acne, allergies, fibromyalgia, chronic fatigue syndrome, halitosis, insomnia, nutritional deficiencies, and AIDS.

7-17. (canceled)

18. The method of claim 1 comprising administering to said individual a GCC agonist, a PDE inhibitor, a MRP4 inhibitor or a MRP5 inhibitor.

19. The method of claim 1 comprising administering to said individual a GCC agonist selected from the group consisting of SEQ ID NOs:2, 3 and 5-58 or a PDE5 inhibitor.

20. The method of claim 3 comprising administering to said individual a GCC agonist, a PDE inhibitor, a MRP4 inhibitor or a MRP5 inhibitor.

21. The method of claim 3 comprising administering to said individual a GCC agonist selected from the group consisting of SEQ ID NOs:2, 3 and 5-58 or a PDE5 inhibitor.

22. (canceled)

23. The method of claim 1 comprising administering to said individual by oral administration a controlled release composition comprising one or more active agents selected from the group consisting of: Guanylyl cyclase C (GCC) agonists, Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that active agent is released and delivered to duodenum, small intestine, large intestine, colon and/or rectum tissue.

24. The method of claim 1 wherein one or more compounds that elevates cGMP is administered in multiple doses.

25. The method of claim 1 wherein one or more compounds that elevates cGMP is administered orally.

26. The method of claim 1 wherein one or more compounds that elevates cGMP is administered orally in an enteric formulation.

27. The method of claim 1 wherein an anti-diarrheal compound or composition is administered in combination with one or more compounds that elevates cGMP.

28. The method of claim 3 comprising administering to said individual by oral administration a controlled release composition comprising one or more active agents selected from the group consisting of: Guanylyl cyclase C (GCC) agonists, Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that active agent is released and delivered to duodenum, small intestine, large intestine, colon and/or rectum tissue.

29. The method of claim 3 wherein one or more compounds that elevates cGMP is administered in multiple doses.

30. The method of claim 3 wherein one or more compounds that elevates cGMP is a administered orally.

31. The method of claim 3 wherein one or more compounds that elevates cGMP is administered orally in an enteric formulation.

32. The method of claim 3 wherein an anti-diarrheal compound or composition is administered in combination with one or more compounds that elevates cGMP.
